(19) United States
(12) Patent Application Publication
(10) Pub. No.: US 2025/0114605 A1
(43) Pub. Date: Apr. 10, 2025

(54) SIDE EFFECT PREDICTION AND EVALUATION FOR NEUROSTIMULATION TREATMENTS

(71) Applicant: Boston Scientific Neuromodulation Corporation, Valencia, CA (US)

(72) Inventors: Lisa Denise Moore, Glendale, CA (US); Benjamin Ray Hoenes, St. Louis Park, MN (US); Leon Mauricio Juarez Paz, Berlin (DE); Mahsa Malekmohammadi, Sherman Oaks, CA (US)

(21) Appl. No.: 18/900,257

(22) Filed: Sep. 27, 2024

Related U.S. Application Data
(60) Provisional application No. 63/543,171, filed on Oct. 9, 2023.

(51) Int. Cl. A61N 1/36; A61N 1/05; A61N 1/372; G16H 10/60; G16H 20/40

(52) U.S. Cl. CPC A61N 1/36135; A61N 1/05; A61N 1/3615; A61N 1/36171; A61N 1/36178; A61N 1/37264; G16H 10/60; G16H 20/40

(57) ABSTRACT

Systems and techniques are disclosed to identify and monitor side effects and treatment outcomes relating to neurostimulation programming. An example technique to identify a side effect relating to neurostimulation programming includes: obtaining neurostimulation programming parameters to be used in a neurostimulation device of a patient; obtaining source data that defines effects of neurostimulation treatment in an anatomical area of the patient; modeling use of the neurostimulation programming parameters in the anatomical area of the neurostimulation treatment, based on the source data, to determine a predicted side effect; and outputting information that identifies characteristics of the predicted side effect.

### CLAIM OF PRIORITY

This application claims the benefit of U.S. Provisional Application No. 63/543,171, filed on Oct. 9, 2023, which is hereby incorporated by reference in its entirety.

### TECHNICAL FIELD

The present disclosure relates generally to data processing in connection with the use of medical devices, and more particularly, to systems, devices, and methods for processing data to predict and respond to treatment outcomes and side effects from implanted electrical stimulation, including neurostimulation treatments used for pain treatment, movement disorders, and/or management of such conditions.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system.

A neurostimulation system can be used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to produce an analgesic effect that masks pain sensation, or to produce a functional effect that allows increased movement or activity of the patient. Other forms of neurostimulation may include a DBS system that uses similar pulses of electricity at particular locations in the brain to reduce symptoms of essential tremor, Parkinson's disease, epilepsy, psychological disorders, or the like.

In connection with a treatment of a particular patient using a neurostimulation system, a variety of data is monitored and collected, including data that is used to drive various forms of closed-loop programming. In order for a programming setting to be deployed to a patient, many use cases require evaluation or testing to ensure that unintended or significant side effects have not and will not occur. However, it may not be feasible or possible to test all possible combinations of programming parameters. Although some combinations of neurostimulation programming settings can be briefly tested in clinical settings, it is often unclear from brief testing whether such programming settings may have latent side effects or will result in unknown adverse outcomes.

### SUMMARY

The following Summary provides examples as an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

Example 1 is a system to identify a side effect relating to neurostimulation programming, the system comprising: one or more processors; and one or more memory devices comprising instructions, which when executed by the one or more processors, cause the one or more processors to perform operations that: obtain neurostimulation programming parameters to be used in a neurostimulation device of a patient; obtain source data that defines effects of neurostimulation treatment in an anatomical area of the patient; model use of the neurostimulation programming parameters in the anatomical area of the neurostimulation treatment, based on the source data, to determine a predicted side effect; and output information that identifies characteristics of the predicted side effect.

In Example 2, the subject matter of Example 1 optionally includes subject matter where the characteristics of the predicted side effect includes a type and severity of the predicted side effect and a timing of the predicted side effect.

In Example 3, the subject matter of any one or more of Examples 1-2 optionally include subject matter where the source data includes clinical effects data collected from the patient, wherein the clinical effects data includes observed data values mapped in multiple dimensions corresponding to neurostimulation parameters, and wherein the predicted side effect is identified based on a proximity or similarity of data values of the neurostimulation programming parameters to the observed data values mapped in the multiple dimensions.

In Example 4, the subject matter of any one or more of Examples 1-3 optionally include subject matter where the operations to model use of the neurostimulation programming parameters include operations that: determine a predicted stimulation volume, based on modeled treatment effects of the neurostimulation programming parameters in the anatomical area indicated by stimulation field modeling; determine a predicted side effect volume, based on modeled side effects of the neurostimulation programming parameters in the anatomical area indicated by the source data; and identify the predicted side effect based on proximity or overlap of the predicted stimulation volume and the predicted side effect volume.

In Example 5, the subject matter of Example 4 optionally includes subject matter where the predicted side effect is selected as a most likely side effect from among a plurality of possible side effects based on the proximity or overlap of the predicted stimulation volume and the predicted side effect volume.

In Example 6, the subject matter of any one or more of Examples 4-5 optionally include subject matter where the source data includes anatomical mapping data that maps the predicted side effect volume to the information that identifies the predicted side effect.

In Example 7, the subject matter of any one or more of Examples 4-6 optionally include subject matter where the anatomical area includes a pathway of neural fibers, wherein the predicted side effect volume corresponds to a neural fiber, and wherein the side effect is predicted based on a proximity of a stimulation lead to the neural fiber, the stimulation lead used to provide the neurostimulation treatment.

In Example 8, the subject matter of any one or more of Examples 1-7 optionally include subject matter where the instructions further cause the one or more processors to perform operations that: cause programming of the neurostimulation device with the neurostimulation programming parameters; wherein the neurostimulation programming parameters are determined based on weights that prioritize treatment of at least one symptom associated with the anatomical area; and wherein the programming is associated with a test of the neurostimulation programming parameters in a program of the neurostimulation device.

In Example 9, the subject matter of Example 8 optionally includes subject matter where the instructions further cause the one or more processors to perform operations that: provide a prompt to a medical user associated with the patient, the prompt including the information that identifies the predicted side effect.

In Example 10, the subject matter of any one or more of Examples 8-9 optionally include subject matter where the instructions further cause the one or more processors to perform operations that: provide a prompt to the patient or a caregiver associated with the patient, the prompt including the information that identifies the predicted side effect; and collect information relating to an observed side effect based on usage of the neurostimulation programming parameters.

In Example 11, the subject matter of Example 10 optionally includes subject matter where the instructions further cause the one or more processors to perform operations that: perform a comparison of the predicted side effect to the observed side effect; and update programming of the neurostimulation device, based on the comparison of the predicted side effect to the observed side effect.

In Example 12, the subject matter of Example 11 optionally includes subject matter where the update to the programming of the neurostimulation device includes use of other programming parameters or programs of the neurostimulation device.

In Example 13, the subject matter of any one or more of Examples 1-12 optionally include subject matter where the neurostimulation programming relates to timing, amplitude, frequency, intensity, duration, pulse patterns, pulse shapes, a spatial location of pulses, waveform shapes, or a spatial location of waveform shapes, of modulated energy provided with at least one lead of the neurostimulation device.

Example 14 is a machine-readable medium including instructions, which when executed by a machine, cause the machine to perform the operations of the system of any of the Examples 1 to 13.

Example 15 is a method to perform the operations of the system of any of the Examples 1 to 13.

Example 16 is a device to identify a side effect relating to neurostimulation programming, the device comprising: one or more processors; and one or more memory devices comprising instructions, which when executed by the one or more processors, cause the one or more processors to: obtain neurostimulation programming parameters to be used in a neurostimulation device of a patient; obtain source data that defines effects of neurostimulation treatment in an anatomical area of the patient; model use of the neurostimulation programming parameters in the anatomical area of the neurostimulation treatment, based on the source data, to determine a predicted side effect; and output information that identifies characteristics of the predicted side effect.

In Example 17, the subject matter of Example 16 optionally includes subject matter where the characteristics of the predicted side effect includes a type and severity of the predicted side effect and a timing of the predicted side effect.

In Example 18, the subject matter of any one or more of Examples 16-17 optionally include subject matter where the source data includes clinical effects data collected from the patient, wherein the clinical effects data includes observed data values mapped in multiple dimensions corresponding to neurostimulation parameters, and wherein the predicted side effect is identified based on a proximity or similarity of data values of the neurostimulation programming parameters to the observed data values mapped in the multiple dimensions.

In Example 19, the subject matter of any one or more of Examples 16-18 optionally include subject matter where to model use of the neurostimulation programming parameters includes to: determine a predicted stimulation volume, based on modeled treatment effects of the neurostimulation programming parameters in the anatomical area indicated by stimulation field modeling; determine a predicted side effect volume, based on modeled side effects of the neurostimulation programming parameters in the anatomical area indicated by the source data; and identify the predicted side effect based on proximity or overlap of the predicted stimulation volume and the predicted side effect volume.

In Example 20, the subject matter of Example 19 optionally includes subject matter where the predicted side effect is selected as a most likely side effect from among a plurality of possible side effects based on the proximity or overlap of the predicted stimulation volume and the predicted side effect volume; and wherein the source data includes anatomical mapping data that maps the predicted side effect volume to the information that identifies the predicted side effect.

In Example 21, the subject matter of Example 20 optionally includes subject matter where the anatomical area includes a pathway of neural fibers, wherein the predicted side effect volume corresponds to a neural fiber, and wherein the side effect is predicted based on a proximity of a stimulation lead to the neural fiber, the stimulation lead used to provide the neurostimulation treatment.

In Example 22, the subject matter of Example 21 optionally includes subject matter where the instructions further cause the one or more processors to: cause programming of the neurostimulation device with the neurostimulation programming parameters; wherein the neurostimulation programming parameters are determined based on weights that prioritize treatment of at least one symptom associated with the anatomical area; and wherein the programming is associated with a test of the neurostimulation programming parameters in a program of the neurostimulation device.

In Example 23, the subject matter of any one or more of Examples 16-22 optionally include subject matter where the instructions further cause the one or more processors to: provide a prompt to a medical user, a patient, or a caregiver associated with the patient, the prompt including the information that identifies the predicted side effect; and collect information relating to an observed side effect based on usage of the neurostimulation programming parameters.

In Example 24, the subject matter of Example 23 optionally includes subject matter where the instructions further cause the one or more processors to: perform a comparison of the predicted side effect to the observed side effect; and update programming of the neurostimulation device, based on the comparison of the predicted side effect to the observed side effect.

In Example 25, the subject matter of any one or more of Examples 16-24 optionally include subject matter where the neurostimulation programming relates to timing, amplitude, frequency, intensity, duration, pulse patterns, pulse shapes, a spatial location of pulses, waveform shapes, or a spatial location of waveform shapes, of modulated energy provided with at least one lead of the neurostimulation device.

Example 26 is a method for identifying a side effect relating to neurostimulation programming, comprising: obtaining neurostimulation programming parameters to be used in a neurostimulation device of a patient; obtaining source data that defines effects of neurostimulation treatment in an anatomical area of the patient; modeling use of the neurostimulation programming parameters in the anatomical area of the neurostimulation treatment, based on the source data, to determine a predicted side effect; and outputting information that identifies characteristics of the predicted side effect.

In Example 27, the subject matter of Example 26 optionally includes subject matter where the characteristics of the predicted side effect includes a type and severity of the predicted side effect and a timing of the predicted side effect.

In Example 28, the subject matter of any one or more of Examples 26-27 optionally include subject matter where the source data includes clinical effects data collected from the patient, wherein the clinical effects data includes observed data values mapped in multiple dimensions corresponding to neurostimulation parameters, and wherein the predicted side effect is identified based on a proximity or similarity of data values of the neurostimulation programming parameters to the observed data values mapped in the multiple dimensions.

In Example 29, the subject matter of any one or more of Examples 26-28 optionally include subject matter where modeling use of the neurostimulation programming parameters includes: determining a predicted stimulation volume, based on modeled treatment effects of the neurostimulation programming parameters in the anatomical area indicated by stimulation field modeling; determining a predicted side effect volume, based on modeled side effects of the neurostimulation programming parameters in the anatomical area indicated by the source data; and identifying the predicted side effect based on proximity or overlap of the predicted stimulation volume and the predicted side effect volume.

In Example 30, the subject matter of Example 29 optionally includes subject matter where the predicted side effect is selected as a most likely side effect from among a plurality of possible side effects based on the proximity or overlap of the predicted stimulation volume and the predicted side effect volume; and wherein the source data includes anatomical mapping data that maps the predicted side effect volume to the information that identifies the predicted side effect.

In Example 31, the subject matter of Example 30 optionally includes subject matter where the anatomical area includes a pathway of neural fibers, wherein the predicted side effect volume corresponds to a neural fiber, and wherein the side effect is predicted based on a proximity of a stimulation lead to the neural fiber, the stimulation lead used to provide the neurostimulation treatment.

In Example 32, the subject matter of Example 31 optionally includes causing programming of the neurostimulation device with the neurostimulation programming parameters; wherein the neurostimulation programming parameters are determined based on weights that prioritize treatment of at least one symptom associated with the anatomical area; and wherein the programming is associated with a test of the neurostimulation programming parameters in a program of the neurostimulation device.

In Example 33, the subject matter of any one or more of Examples 26-32 optionally include providing a prompt to a medical user, a patient, or a caregiver associated with the patient, the prompt including the information that identifies the predicted side effect; and collecting information relating to an observed side effect based on usage of the neurostimulation programming parameters.

In Example 34, the subject matter of Example 33 optionally includes performing a comparison of the predicted side effect to the observed side effect; and updating programming of the neurostimulation device, based on the comparison of the predicted side effect to the observed side effect.

In Example 35, the subject matter of any one or more of Examples 26-34 optionally include subject matter where the neurostimulation programming relates to timing, amplitude, frequency, intensity, duration, pulse patterns, pulse shapes, a spatial location of pulses, waveform shapes, or a spatial location of waveform shapes, of modulated energy provided with at least one lead of the neurostimulation device.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, an embodiment of a neurostimulation system.

FIG. 2 illustrates, by way of example, an embodiment of a stimulation device and a lead system, such as may be implemented in the neurostimulation system of FIG. 1.

FIG. 3 illustrates, by way of example, an embodiment of a programming device, such as may be implemented in the neurostimulation system of FIG. 1.

FIG. 4 illustrates, by way of example, an implantable neurostimulation system and portions of an environment in which the system may be used.

FIG. 5 illustrates, by way of example, an embodiment of an implantable stimulator and one or more leads of a neurostimulation system, such as the implantable neurostimulation system of FIG. 4.

FIG. 6 illustrates, by way of example, an embodiment of a programming system and patient data analysis system for use with a neurostimulation system, such as the implantable neurostimulation system of FIG. 4.

FIG. 7 illustrates, by way of example, an embodiment of data processing systems and user interfaces used in connection with prediction and monitoring of side effects for neurostimulation treatments.

FIG. 8 illustrates, by way of example, an estimation of neurostimulation side effects based on data value modeling.

FIG. 9A illustrates, by way of example, an estimation of neurostimulation side effects based on stimulation field modeling.

FIG. 9B illustrates, by way of example, an estimation of neurostimulation side effects on fiber tracts based on stimulation field modeling.

FIG. 10 illustrates, by way of example, a data processing flow for side effect monitoring of neurostimulation treatment in a clinical setting.

FIG. 11 illustrates, by way of example, a data processing flow for side effect monitoring of neurostimulation treatment in a patient setting.

FIG. 12 illustrates, by way of example, a closed-loop data processing flow for neurostimulation treatment incorporating side effect prediction and observation.

FIG. 13 illustrates, by way of example, a flowchart of a method implemented by a system or device to predict side effects in connection with neurostimulation programming.

FIG. 14 illustrates, by way of example, a block diagram of an embodiment of a computing system for performing patient data analysis in connection with the side effects data processing operations discussed herein.

FIG. 15 illustrates, by way of example, a block diagram of an embodiment of a computing system implementing neurostimulation programming circuitry, to cause programming of an implantable electrical neurostimulation device.

FIG. 16 is a block diagram illustrating a machine in the example form of a computer system, within which a set or sequence of instructions may be executed to cause the machine to perform any one of the methodologies discussed herein, according to an example embodiment.

### DETAILED DESCRIPTION

This document discusses various techniques for the collection, processing, storage, and communication of data relevant to treatment with an implantable electrical neurostimulation device. Such data includes condition symptoms, treatment side effects, treatment outcome data, anatomy targets and stimulation effects, and related predictions and workflows involving such symptoms and side effects. As an example, various systems and methods are described to identify most probable side effects that would occur in a particular patient in response to use of a particular programming setting, such as a programming setting (or combination of settings) that has not been tested or fully tested with the particular patient or on a larger patient population. Such side effects may be predicted based on, among other values, a position of the estimated field of neurostimulation relative to anatomical targets and previously tested stimulation settings.

In some existing approaches of neurostimulation treatment, in order for a setting to be stored and available for use by a patient, it must have been tested at least briefly. However, some side effects may not emerge until some later time, such as 30 minutes or more after stimulation has been applied. Additionally, some side effects may not be particularly disruptive to the patient or may involve psychological symptoms, so side effects may not be readily observable during brief testing. Additionally, based on the variety of physiological and psychological effects that may occur from neurostimulation treatment, a clinician or caregiver may not know what type of a side effect to look for. As closed-loop systems provide additional optimization and exploration of new programming settings and program usage, the consideration of delayed and latent side effects will become very important to ensure neurostimulation safety and efficacy.

In various examples, approaches are described for the identification of side effects and accompanying probabilities, rankings, and observations. In a first example, potential side effects may be identified based on clinical effects data (CED), such as CED that has been captured for a patient that maps side effects occurring from multiple dimensions of data values observed during neurostimulation operation. For instance, if clinical effects data has been captured for a patient that suggests adverse side effects with previously tested amplitude and lead level positions, then new values that are close to the previously tested amplitude and positions may have a high likelihood of causing similar side effects.

In a second example, potential side effects may be identified based on anatomical data and neurostimulation electrode placement. For example, in the event that a predicted stimulation field from an electrode overlaps or is close to specific anatomical structures or regions that are associated with a particular side effect, then the occurrence of this particular side effect may be automatically suggested and tracked.

In a third example, anatomical data that is evaluated for potential side effects may be enhanced by the modeling of a neurostimulation activation of a neural fiber tract, including fiber tracts that reach structures or regions associated with a given side effect. Thus, in addition to effects from basic anatomical area tracking, side effects that occur from an activated fiber (e.g., a collection of neural fibers that intersects with a given stimulation field modeling volume) may be automatically suggested and tracked.

In a fourth example, various treatment approaches for multiple symptoms are automatically determined and prioritized, based on symptom weighting, side effect prediction, and predicted outcomes. For example, a data processing system may be adapted to determine symptom significance and weighting using one or a series of measurements collected from the patient (e.g., at home) through the assistance of a mobile application. The discovered symptoms and weights can supplement or replace in clinic testing, and can enable a closed-loop approach that targets symptoms of significance in the patient that are expected or predicted to respond to treatment.

The following discussion provides an introduction to the features of an example neurostimulation system and how a neurostimulation system is programmed to cause specific neurostimulation effects on a subject patient. Thereafter, the following discusses how the results of these neurostimulation effects, including problematic side effects, may be predicted and mitigated as a result of patient-specific neurostimulation programming. Finally, various examples are provided based on the use of symptom weighting and side-effect predictions, for program adjustment and various clinical and patient warnings, loggings, and follow up actions.

While neurostimulation therapies, such as SCS and DBS therapies, are specifically discussed as examples, the present subject matter may apply to other therapies that employ stimulation pulses of electrical or other forms of energy for treating chronic pain or similar physiological or medical conditions.

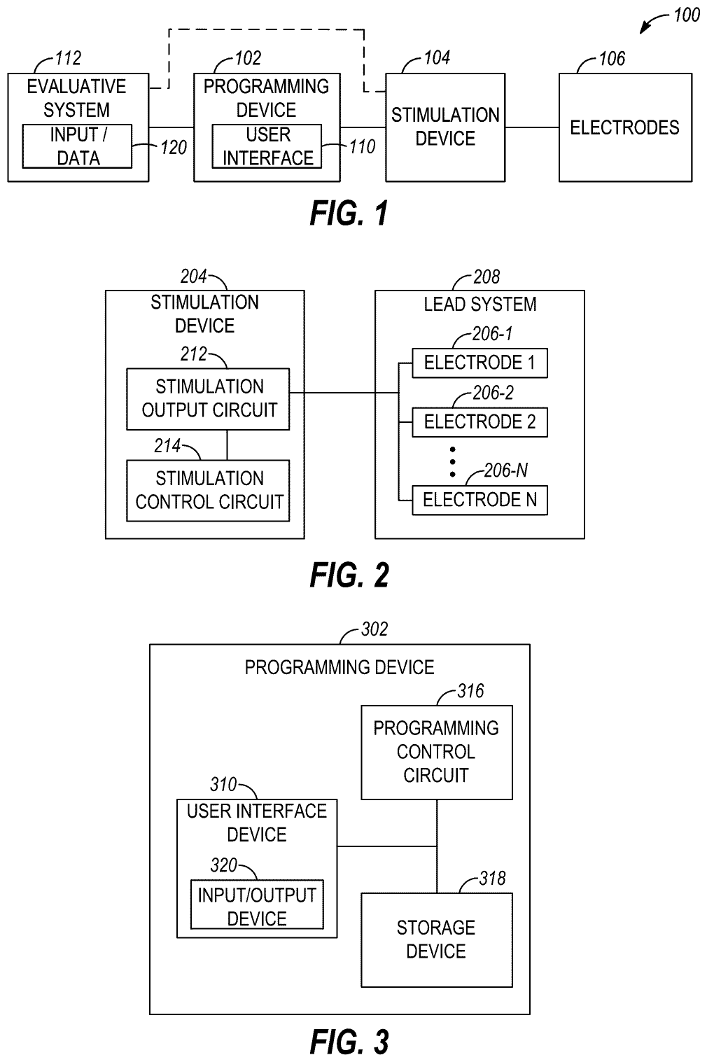

FIG. 1 illustrates an embodiment of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are selected or programmable by a clinical user, such as a physician or other caregiver who treats the patient using system 100; however, some of the parameters may also be provided in connection with automated (e.g., closed-loop or partially-closed-loop) programming logic and adjustment. Programming device 102 provides the user with accessibility to implement, change, or modify the programmable parameters. In various embodiments, programming device 102 is configured to be communicatively coupled to stimulation device 104 via a wired or wireless link.

In various embodiments, programming device 102 includes a user interface 110 (e.g., a user interface embodied by a graphical, text, voice, or hardware-based user interface) that allows the user to set and/or adjust values of the user-programmable parameters by creating, editing, loading, and removing programs that include parameter combinations such as patterns and waveforms. Such waveforms may include, for example, the waveform of a pattern of neurostimulation pulses to be delivered to the patient as well as individual waveforms that are used as building blocks of the pattern of neurostimulation pulses. Examples of such individual waveforms include pulses, pulse groups, and groups of pulse groups. The program and respective sets of parameters may also define an electrode selection specific to each individually defined waveform.

The neurostimulation energy that is discussed herein may be delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Many current neurostimulation systems are programmed to deliver periodic pulses with one or a few uniform waveforms continuously or in bursts. However, neural signals may include more sophisticated patterns to communicate various types of information, including sensations of pain, pressure, temperature, etc.

The present approaches further provide examples of an evaluative system 112, such as a patient data analysis system, which is used to analyze side effects based on clinical or anatomical data 120 related to a prior, ongoing, or planned neurostimulation treatment. This evaluative system 112 can initiate a data processing action related to programming and effects of the neurostimulation treatment based on analysis performed on the patient input or anatomical data 120. The clinical input provided by testing the patient or anatomical data 120 may be collected from the patient, a clinician or other medical provider, a caregiver, another third party, or by sensors and data monitoring devices, and then analyzed by the evaluative system 112. The evaluative system 112 may reside at a remote computing system, such as a cloud server that provides services to perform side effect data processing on demand when invoked by the user interface 110 or other entities. In various examples, in addition to modeling side effects from anatomical data and clinical effects data, the evaluative system 112 may also directly or indirectly collect information regarding the patient or the ongoing neurostimulation treatment by being communicatively coupled with the programming device 102 or the stimulation device 104.

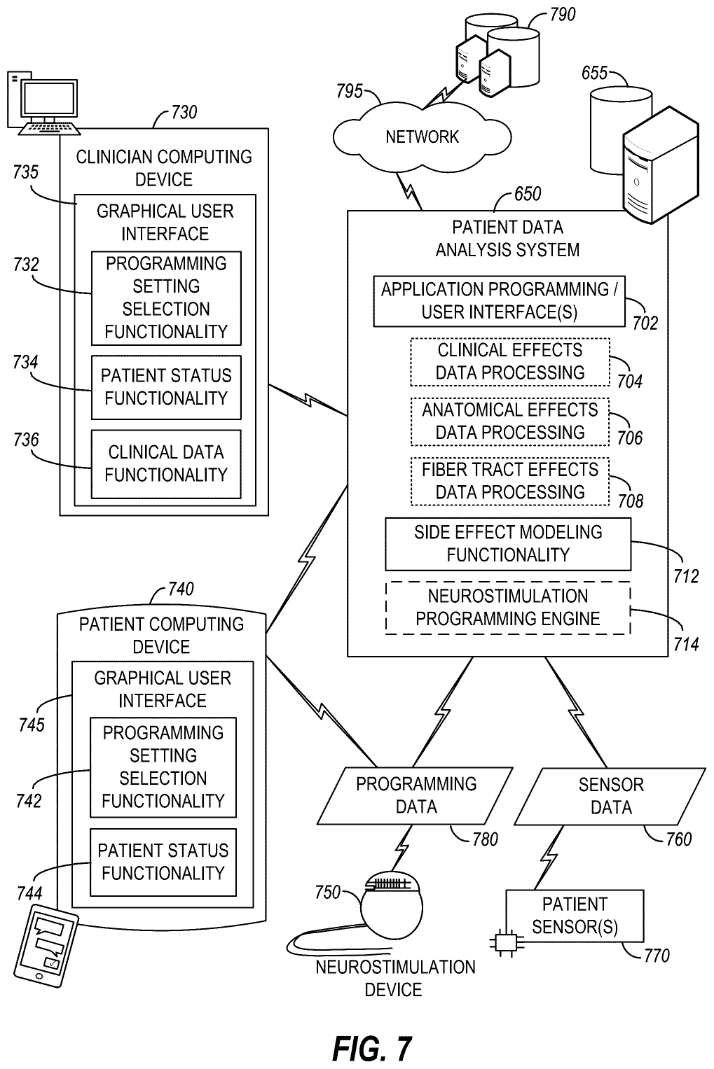

As described in more detail below with respect to the data flows in FIGS. 7 to 13, the evaluative system 112 may assist with the collection of patient source data such as anatomy data and clinical effects data, to obtain information relevant to the beneficial effects or adverse effects (i.e., side effects) of the neurostimulation treatment by the stimulation device 104. The evaluative system 112 may perform one or more actions to identify, model, classify, forecast, or predict side effects or patient symptoms, including with the use of artificial intelligence other algorithmic processing. The evaluative system 112 may further initiate or control workflows that prompt and measure whether treatment side effects or benefits have occurred, and to provide remedial programming adjustments based on the amount or type of the side effects.

Example parameters that can be implemented by a selected neurostimulation program include, but are not limited to the following: amplitude, pulse width, frequency, duration, total charge injected per unit time, cycling (e.g., on/off time), pulse shape, number of phases, phase order, interphase time, charge balance, ramping, as well as spatial variance (e.g., electrode configuration changes over time). As detailed in FIG. 6, a controller, e.g., controller 630 of FIG. 6, can implement program(s) and parameter setting(s) to affect a specific neurostimulation waveform, pattern, or energy output, using a program or setting in storage, e.g., external storage device 616 of FIG. 6, or using settings communicated via an external communication device 618 of FIG. 6 corresponding to the selected program. The implementation of such program(s) or setting(s) may further define a therapy strength and treatment type corresponding to a specific pulse group, or a specific group of pulse groups, based on the specific programs or settings. The evaluative system 112 and the evaluation of the patient input or anatomical data 120 provides a mechanism to determine the effectiveness or safety of such programs or settings for a particular patient, and to classify treatment side effects and associated outcomes in an indirect or direct manner.

Portions of the evaluative system 112, the stimulation device 104 (e.g., implantable medical device), or the programming device 102 can be implemented using hardware, software, or any combination of hardware and software. Portions of the stimulation device 104 or the programming device 102 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. The system 100 could also include a subcutaneous medical device (e.g., subcutaneous ICD, subcutaneous diagnostic device), wearable medical devices (e.g., patch-based sensing device), or other external medical devices.

FIG. 2 illustrates an embodiment of a stimulation device 204 and a lead system with one or more leads 208, such as may be implemented in neurostimulation system 100 of FIG. 1. Stimulation device 204 represents an embodiment of stimulation device 104 and includes a stimulation output circuit 212 and a stimulation control circuit 214. Stimulation output circuit 212 produces and delivers neurostimulation pulses, including the neurostimulation waveform and parameter settings implemented via a program selected or implemented with the user interface 110. Stimulation control circuit 214 controls the delivery of the neurostimulation pulses using the plurality of stimulation parameters, which specifies a pattern of the neurostimulation pulses. Lead system includes one or more leads 208 each configured to be electrically connected to stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses.

In various embodiments, the number of leads and the number of electrodes on each lead depend on, for example, the distribution of target(s) of the neurostimulation and the need for controlling the distribution of electric field at each target. In one embodiment, the lead system includes 2 leads each having 8 electrodes. Those of ordinary skill in the art will understand that the neurostimulation system 100 may include additional components such as sensing circuitry for patient monitoring and/or feedback control of the therapy, telemetry circuitry, and power. The neurostimulation system 100 may also integrate with other sensors, or such other sensors may independently provide information for use with programming of the neurostimulation system 100, and for data collection and evaluation (e.g., to be considered as part of side effect or treatment efficacy tracking).

The neurostimulation system 100 may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. Other parameters that may be controlled or varied include the amplitude, pulse width, and rate (or frequency) of the electrical pulses. Each electrode configuration, along with the electrical pulse parameters, can be referred to as a “modulation parameter” set. Each set of modulation parameters, including fractionalized current distribution to the electrodes (as percentage cathodic current, percentage anodic current, or off), may be stored and combined into a program that can then be used to modulate multiple regions within the patient.

The neurostimulation system 100 may be configured to deliver different electrical fields to achieve a temporal summation of modulation. The electrical fields can be generated respectively on a pulse-by-pulse basis. For example, a first electrical field can be generated by the electrodes (using a first current fractionalization) during a first electrical pulse of the pulsed waveform, a second different electrical field can be generated by the electrodes (using a second different current fractionalization) during a second electrical pulse of the pulsed waveform, a third different electrical field can be generated by the electrodes (using a third different current fractionalization) during a third electrical pulse of the pulsed waveform, a fourth different electrical field can be generated by the electrodes (using a fourth different current fractionalized) during a fourth electrical pulse of the pulsed waveform, and so forth. These electrical fields can be rotated or cycled through multiple times under a timing scheme, where each field is implemented using a timing channel. The electrical fields may be generated at a continuous pulse rate, or as bursts of pulses. Furthermore, the interpulse interval (i.e., the time between adjacent pulses), pulse amplitude, and pulse duration during the electrical field cycles may be uniform or may vary within the electrical field cycle. In some examples, the modulation field may be shaped to enhance modulation of some neural structures and diminish modulation at other neural structures. The modulation field may be shaped by using multiple independent current control (MICC) or multiple independent voltage control to guide the estimate of current fractionalization among multiple electrodes and estimate a total amplitude that provide a desired strength. For example, the modulation field may be shaped to enhance the modulation of dorsal horn neural tissue and to minimize the modulation of dorsal column tissue.

FIG. 3 illustrates an embodiment of a programming device 302, such as may be implemented in neurostimulation system 100. Programming device 302 represents an embodiment of programming device 102 and includes a storage device 318, a programming control circuit 316, and a user interface device 310. Programming control circuit 316 generates the plurality of stimulation parameters that controls the delivery of the neurostimulation pulses according to the pattern of the neurostimulation pulses. The user interface device 310 represents an embodiment to implement the user interface 110.

In various embodiments, the user interface device 310 includes an input/output device 320 that is capable to receive user interaction and commands to load, modify, and implement neurostimulation programs and schedule delivery of the neurostimulation programs. In various embodiments, the input/output device 320 allows the user to create, establish, access, and implement respective parameter values of a neurostimulation program through graphical selection (e.g., in a graphical user interface output with the input/output device 320), or other graphical input/output relating to therapy objectives, efficacy of applied treatment, user feedback, and the like. In various examples, the user interface device 310 can receive user input to initiate or control the implementation of the programs or program changes which are recommended, modified, selected, or loaded through use of an open or closed loop programming system.

In various embodiments, the input/output device 320 allows a user (e.g., a patient user or, a medical user) to apply, change, modify, or discontinue certain building blocks of a program and a frequency at which a selected program is delivered. In various embodiments, the input/output device 320 can allow the user to save, retrieve, and modify programs (and program settings), such as from programs that are loaded from a clinical encounter or pre-programmed (e.g., as templates). In various embodiments, the input/output device 320 and accompanying software on the user interface device 310 allows newly created building blocks, program components, programs, and program modifications to be saved, stored, or otherwise persisted in storage device 318. Thus, it will be understood that the user interface device 310 may allow many forms of device operation and control, even if automated (e.g., closed loop) programming is occurring.

The user interface device 310 may provide an interactive mechanism, controllable with the input/output device 320, for the entry or indication of determined side effects and side effects data (including, clinical effects data that may have been observed by or for the patient). In one embodiment, the input/output device 320 includes a touchscreen. In various embodiments, the input/output device 320 includes any type of presentation device, such as interactive or non-interactive screens, and any type of user input device that allows the user to interact with a user interface to implement, remove, or schedule the programs. Thus, the input/output device 320 may include one or more of a touchscreen, keyboard, keypad, touchpad, trackball, joystick, and mouse. The logic of the user interface 110, the stimulation control circuit 214, and the programming control circuit 316, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

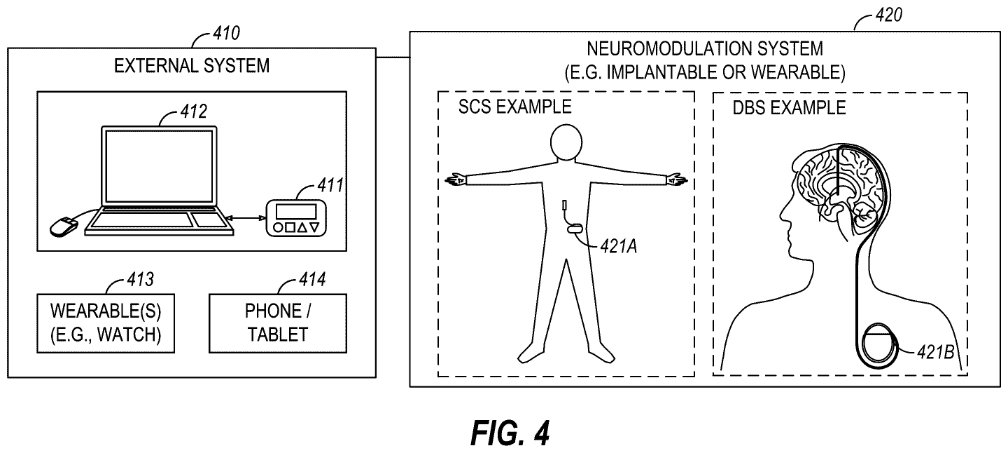

FIG. 4 illustrates an implantable neurostimulation system 420 and portions of an environment in which system 420 may be used, such as may be implemented as the stimulation device 104, 204 illustrated in FIGS. 1 and 2. FIG. 4 specifically illustrates, by way of example and not limitation, the neurostimulation system 100 of FIG. 1 implemented in a spinal cord stimulation (SCS) system or a deep brain stimulation (DBS) system. The illustrated neuromodulation system 420 connects with an external system 410 that may include at least one programming device. The illustrated external system 410 may include a programmer 412 (e.g., clinician programmer) configured for use by a clinician to communicate with and program the neurostimulator, and a remote control device 411 configured for use by the patient to communicate with and program the neurostimulator. For example, the remote control device 411 may allow the patient to turn a therapy on and off and/or may allow the patient to adjust patient-programmable parameter(s) of the plurality of modulation parameters (e.g., by switching programs).

FIG. 4 further illustrates the neuromodulation system 420 as an ambulatory medical device, such as implemented by stimulation device 421A or stimulation device 421B. Examples of ambulatory devices include wearable or implantable neuromodulators. The external system 410 may include a network of computers, including computer(s) remotely located from the ambulatory medical device that are capable of communicating via one or more communication networks with the programmer 412 and/or the remote control device 411. The remotely located computer(s) and the ambulatory medical device may be configured to communicate with each other via another external device such as the programmer 412 or the remote control device 411.

The external system 410 may also include one or more wearables 413 and a portable device 414 such as a smartphone or tablet. In some examples, the wearables 413 and the portable device 414 may allow a user to obtain and provide input data, such as sensor data values (e.g., from a physiologic sensor of a wearable) or feedback/status information (e.g., on a phone/tablet screen) in connection with a data collection process. In some examples, the remote control device 411 and/or the programmer 412 also may display recommendations or program settings (e.g., derived from recommendations as part of a fully or partially closed-loop programming process). The remote control device 411 and/or the programmer 412 may be used to communicate other aspects of input and output, including inputs from the usage data of various neurostimulation programs, events associated with such programs, and the like.

As used herein, the terms “neurostimulator,” “stimulator,” “neurostimulation,” and “stimulation” generally refer to the delivery of electrical energy that affects the neuronal activity of neural tissue, which may be excitatory or inhibitory; for example by initiating an action potential, inhibiting or blocking the propagation of action potentials, affecting changes in neurotransmitter/neuromodulator release or uptake, and inducing changes in neuro-plasticity or neurogenesis of tissue. It will be understood that other clinical effects and physiological mechanisms may also be provided through use of such stimulation techniques.

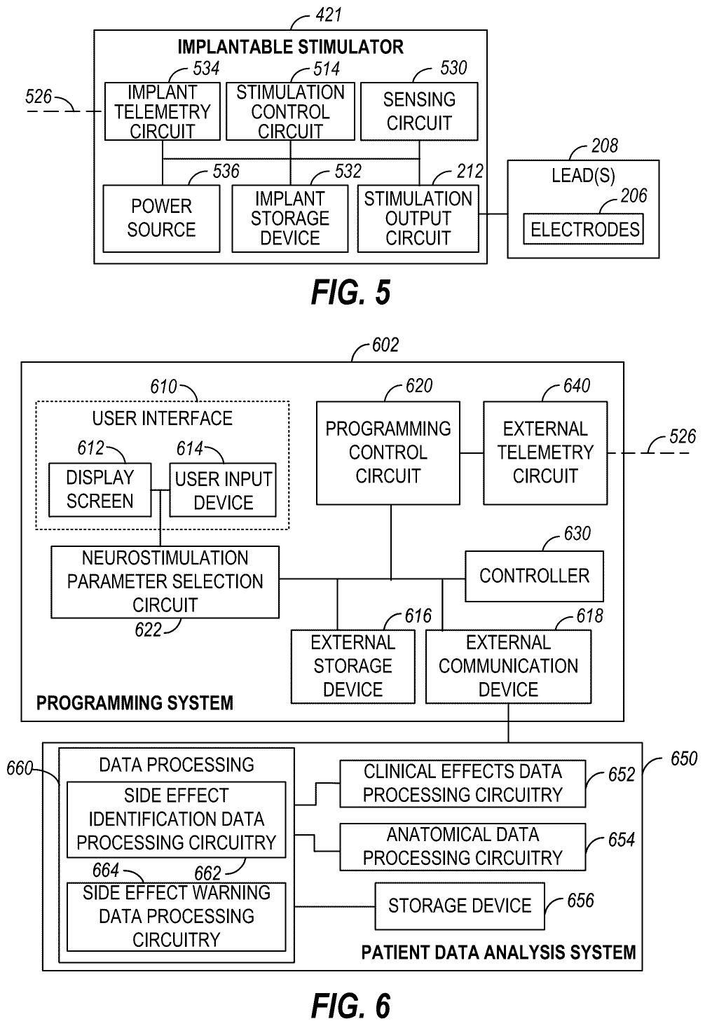

FIG. 5 illustrates an embodiment of the implantable stimulator 421 and the one or more leads 208 of an implantable neurostimulation system, such as the implantable system 420. The implantable stimulator 421 may include a sensing circuit 530 used for an optional sensing capability, stimulation output circuit 212, a stimulation control circuit 514, an implant storage device 532, an implant telemetry circuit 534, and a power source 536. The sensing circuit 530, when included and needed, senses one or more physiological signals for purposes of patient monitoring and/or feedback control of the neurostimulation. Examples of the one or more physiological signals includes neural and other signals each indicative of a condition of the patient that is treated by the neurostimulation and/or a response of the patient to the delivery of the neurostimulation.

The stimulation output circuit 212 is electrically connected to electrodes 206 through the one or more leads 208, and delivers each of the neurostimulation pulses through a set of electrodes selected from the electrodes 206. The stimulation output circuit 212 can implement, for example, the generating and delivery of a customized neurostimulation waveform (e.g., implemented from a parameter of a selected or specified program) to an anatomical target of a patient.

The stimulation control circuit 514 represents an embodiment of the stimulation control circuit 214 and controls the delivery of the neurostimulation pulses using the plurality of stimulation parameters specifying the pattern of the neurostimulation pulses. In one embodiment, the stimulation control circuit 514 controls the delivery of the neurostimulation pulses using the one or more sensed physiological signals and processed input from patient feedback interfaces. The implant telemetry circuit 534 provides the implantable stimulator 421 with wireless communication with another device such as a device of the external system 410, including receiving values of the plurality of stimulation parameters from the external system 410. The implant storage device 532 stores values of the plurality of stimulation parameters, including parameters from one or more programs which are activated, de-activated, or modified using the approaches discussed herein.

The power source 536 provides the implantable stimulator 421 with energy for its operation. In one embodiment, the power source 536 includes a battery. In one embodiment, the power source 536 includes a rechargeable battery and a battery charging circuit for charging the rechargeable battery. The implant telemetry circuit 534 may also function as a power receiver that receives power transmitted from external system 410 through an inductive couple.

In various embodiments, the sensing circuit 530, the stimulation output circuit 212, the stimulation control circuit 514, the implant telemetry circuit 534, the implant storage device 532, and the power source 536 are encapsulated in a hermetically sealed implantable housing. In various embodiments, the lead(s) 208 are implanted such that the electrodes 206 are placed on and/or around one or more targets to which the neurostimulation pulses are to be delivered, while the implantable stimulator 421 is subcutaneously implanted and connected to the lead(s) 208 at the time of implantation.

FIG. 6 illustrates an embodiment of a programming system 602 used as part of an implantable neurostimulation system, such as the external system 420, with the programming system 602 configured to send and receive device data (e.g., commands, parameters, program selections, information). FIG. 6 also illustrates an embodiment of a patient data analysis system 650, communicatively coupled to the programming system 602. The patient data analysis system 650 is used to evaluate patient symptoms, to identify treatment outcomes, and to provide warnings of side effects (and, optionally trigger remedial actions) in connection with planned or ongoing neurostimulation treatment by the implantable neurostimulation system.

The programming system 602 represents an embodiment of the programming device 302, and includes an external telemetry circuit 640, an external storage device 616, a programming control circuit 620, a user interface device 610, a controller 630, and an external communication device 618, to effect programming of a connected neurostimulation device. The operation of the neurostimulation parameter selection circuit 622 enables selection, modification, and implementation of a particular set of parameters or settings for neurostimulation programming (e.g., via selection of a program, specification by a closed-loop or open-loop programming process, use by a patient or clinician for testing or experimentation, or the like).

The external telemetry circuit 640 provides the programming system 602 with wireless communication to and from another controllable device such as the implantable stimulator 421 via a telemetry link 526, including transmitting one or a plurality of stimulation parameters (including selected, identified, or modified stimulation parameters of a selected program) to the implantable stimulator 421. In one embodiment, the external telemetry circuit 640 also transmits power to the implantable stimulator 421 through inductive coupling.

The external communication device 618 may provide a mechanism to conduct communications with a programming information source, such as a data service, program modeling system, to receive program information, settings and values, models, functionality controls, or the like, via an external communication link (not shown). In a specific example, the external communication device 618 communicates with the patient data analysis system 650 to identify parameters or settings that are selected, modified, or implemented in the neurostimulation programming. The external communication device 618 may communicate using any number of wired or wireless communication mechanisms described in this document, including but not limited to IEEE 802.11 (Wi-Fi), Bluetooth, Infrared, and like standardized and proprietary wireless communications implementations. Although the external telemetry circuit 640 and the external communication device 618 are depicted as separate components within the programming system 602, the functionality of both of these components may be integrated into a single communication chipset, circuitry, or device.

The external storage device 616 stores a plurality of existing neurostimulation waveforms, including definable waveforms for use as a portion of the pattern of the neurostimulation pulses, settings and setting values, other portions of a program, and related treatment information. In various embodiments, each waveform of the plurality of individually definable waveforms includes one or more pulses of the neurostimulation pulses, and may include one or more other waveforms of the plurality of individually definable waveforms. Examples of such waveforms include pulses, pulse blocks, pulse trains, and train groupings, and programs. The existing waveforms stored in the external storage device 616 can be definable at least in part by one or more parameters including, but not limited to the following: amplitude, pulse width, frequency, duration(s), electrode configurations, total charge injected per unit time, cycling (e.g., on/off time), waveform shapes, spatial locations of waveform shapes, pulse shapes, number of phases, phase order, interphase time, charge balance, and ramping.

The external storage device 616 may also store a plurality of individually definable fields that may be implemented as part of a program. Each waveform of the plurality of individually definable waveforms is associated with one or more fields of the plurality of individually definable fields. Each field of the plurality of individually definable fields is defined by one or more electrodes of the plurality of electrodes through which a pulse of the neurostimulation pulses is delivered and a current distribution of the pulse over the one or more electrodes. A variety of settings in a program may be correlated to the control of these waveforms and definable fields.

The programming control circuit 620 represents an embodiment of a programming control circuit 316 and may translate or generate the specific stimulation parameters or changes which are to be transmitted to the implantable stimulator 421, based on the results of the neurostimulation parameter selection circuit 622. The pattern may be defined using one or more waveforms selected from the plurality of individually definable waveforms (e.g., defined by a program) stored in an external storage device 616. In various embodiments, the programming control circuit 620 checks values of the plurality of stimulation parameters against safety rules to limit these values within constraints of the safety rules. In one embodiment, the safety rules are heuristic rules.

The user interface device 610 represents an embodiment of the user interface device 310 and allows the user (including a patient or clinician) to provide input relevant to therapy objectives, such as to switch programs or change operational use of the programs. The user interface device 610 includes a display screen 612, a user input device 614, and may implement or couple to the neurostimulation parameter selection circuit 622, or data provided from the patient data analysis system 650. The display screen 612 may include any type of interactive or non-interactive screens, and the user input device 614 may include any type of user input devices that supports the various functions discussed in this document, such as a touchscreen, keyboard, keypad, touchpad, trackball, joystick, and mouse. The user interface device 610 may also allow the user to perform other functions where user interface input is suitable (e.g., to select, modify, enable, disable, activate, schedule, or otherwise define a program, sets of programs, provide feedback or input values, or perform other monitoring and programming tasks). Although not shown, the user interface device 610 may also generate a visualization of such characteristics of device implementation or programming, and receive and implement commands to implement or revert the program and the neurostimulator operational values (including a status of implementation for such operational values). These commands and visualization may be performed in a review and guidance mode, testing or experimentation mode, status mode, or in a real-time programming mode. Consistent with the examples provided herein, the user interface device 610 may invoke or output the results of symptom or side effect modeling using the processes discussed herein (e.g., with reference to FIGS. 7 to 13).

The controller 630 can be a microprocessor that communicates with the external telemetry circuit 640, the external communication device 618, the external storage device 616, the programming control circuit 620, the neurostimulation parameter selection circuit 622, and the user interface device 610, via a bidirectional data bus. The controller 630 can be implemented by other types of logic circuitry (e.g., discrete components or programmable logic arrays) using a state machine type of design. As used in this disclosure, the term “circuitry” should be taken to refer to discrete logic circuitry, firmware, or to the programming of a microprocessor.

The patient data analysis system 650 is configured to operate data processing circuitry 660, which may include side effect identification data processing circuitry 662 that identifies properties (e.g., type, timing, location) of one or more neurostimulation treatment side effects and side effect warning data processing circuitry 664 that provides output such as warnings or informative details related to the treatment side effects. The side effect identification data processing circuitry 662 may model the use of particular neurostimulation programming parameters (and parameter values) using source data related to neurostimulation clinical effects, such as clinical effects data provided by clinical effects data processing circuitry 652 (e.g., data values from treatment observations from the patient or a population of patients) or neuro-anatomical data provided by anatomical data input processing circuitry 654 (e.g., patient-specific or general population anatomical data).

The patient data analysis system 650 also is depicted as including a storage device 656 to store or persist data related to the side effect identification or warning, and for associated medical data, device data, patient or clinician input and output, and related settings, logic, or algorithms. Other hardware features of the patient data analysis system 650 are not depicted for simplicity, but are suggested from functional capabilities and operations in the following figures.

FIG. 7 illustrates, by way of example, an embodiment of data interactions among the patient data analysis system 650, a clinician computing device 730, and a patient computing device 740, for the collection and processing of data related to side effects from neurostimulation treatment. Here, data related to side effects and treatment outcomes can be evaluated and processed for a particular patient being treated (or to be treated) with neurostimulation via a neurostimulation device 750, based on the past, current, or future (e.g., predicted) use of programming parameters in programming data 780.

At a high level, the patient data analysis system 650 identifies effects of the neurostimulation treatment for a patient based on source data representing clinical effects, anatomical effects, neural fiber tract effects, or derivatives of such data. This source data is respectively depicted in the patient data analysis system 650 as originating from clinical effects data (processed in engine 704), anatomical effects data (processed in engine 706), and fiber tract effects data (processed in engine 708). The patient data analysis system 650 evaluates this data using a side effect modeling functionality 712, to model use of particular neurostimulation programming parameters to predict possible side effects.

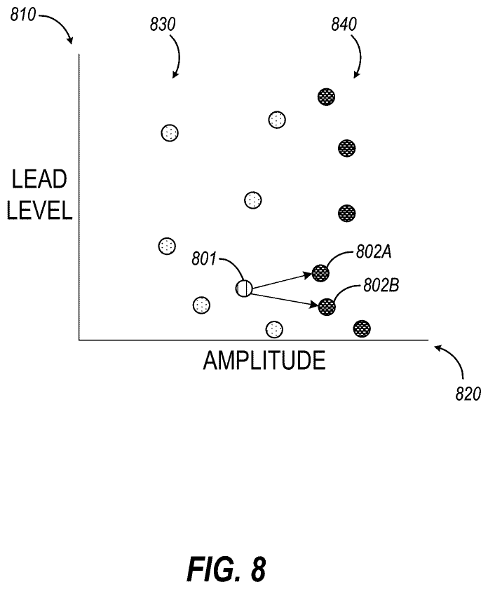

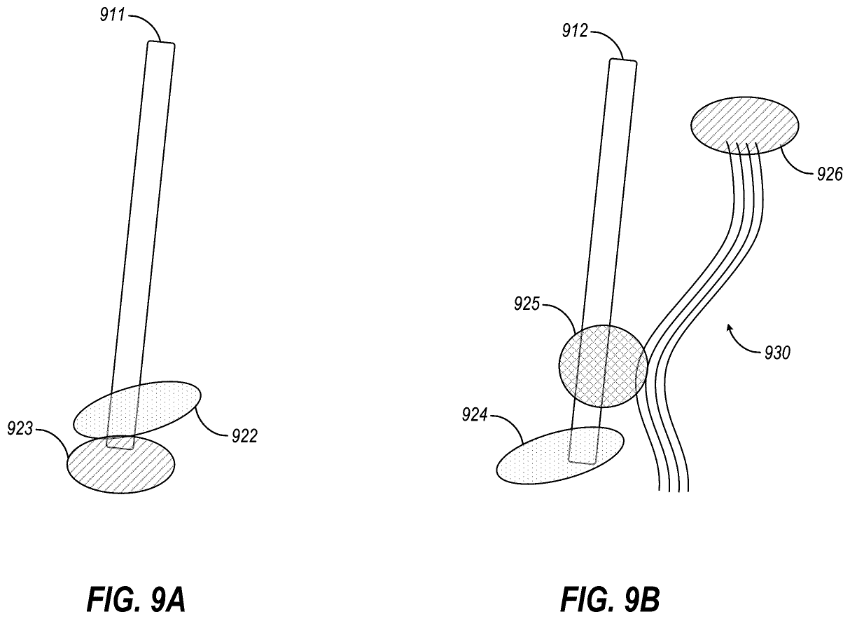

The clinical effects data processing engine 704 may evaluate patient-specific or patient population data from among multiple sources of clinical effects data, including clinical effects that indicates prior symptoms or effects from the use of particular programming parameters and neurostimulation locations (e.g., as discussed with reference to FIG. 8, below). The anatomical effects data processing engine 706 may evaluate patient-specific or population data from among multiple sources of anatomical data, including neurostimulation effect atlases or mappings (e.g., as discussed with reference to FIG. 9A, below). The fiber tract effects data processing engine 708 may evaluate patient-specific or population data from among multiple sources of anatomical data relating to neural fibers, including fiber measurements, characteristics, or correlations between fiber activation from neurostimulation and particular side effects (e.g., as discussed with reference to FIG. 9B, below).

The side effect modeling functionality 712 may identify, classify, generate, or output a predicted side effect (or multiple predicted side effects), and associated properties such as: probability/likelihood of the side effect, a type of the side effect (e.g., paresthesia, pain, emotional effects), timing of the side effect (including the amount of time to first appear, or the duration of time that the symptoms last), location(s) of the side effect, characteristics of affected physiological systems or second order effects, and the like. The side effect modeling functionality 712 may be used to determine whether a particular programming parameter, set of programming parameters, or program is tolerable and effective to test or to use. The side effect modeling functionality 712 also may be used to determine if latent or nascent side effects have developed, and whether changes to patient programming may result in a reduction or removal of side effects.

The side effects that are identified and tracked may be maintained with data values in local storage (e.g., a database, maintained on storage device 656) or in remote storage (e.g., remote system 790 connected via network 795). The patient data analysis system 650 may operate various types of algorithms, models, or rule sets to interpret and convert patient data related to side effects, including specifically identified data values relating to multiple dimensions and characteristics of neurostimulation treatments and programming parameters. The patient data analysis system 650 may receive or obtain data via an interface, such as provided from an application programming interface or user interface 702. The patient data analysis system 650 may directly or indirectly communicate with the clinician computing device 730, the patient computing device 740, or third party devices and platforms not depicted, to obtain and receive data.

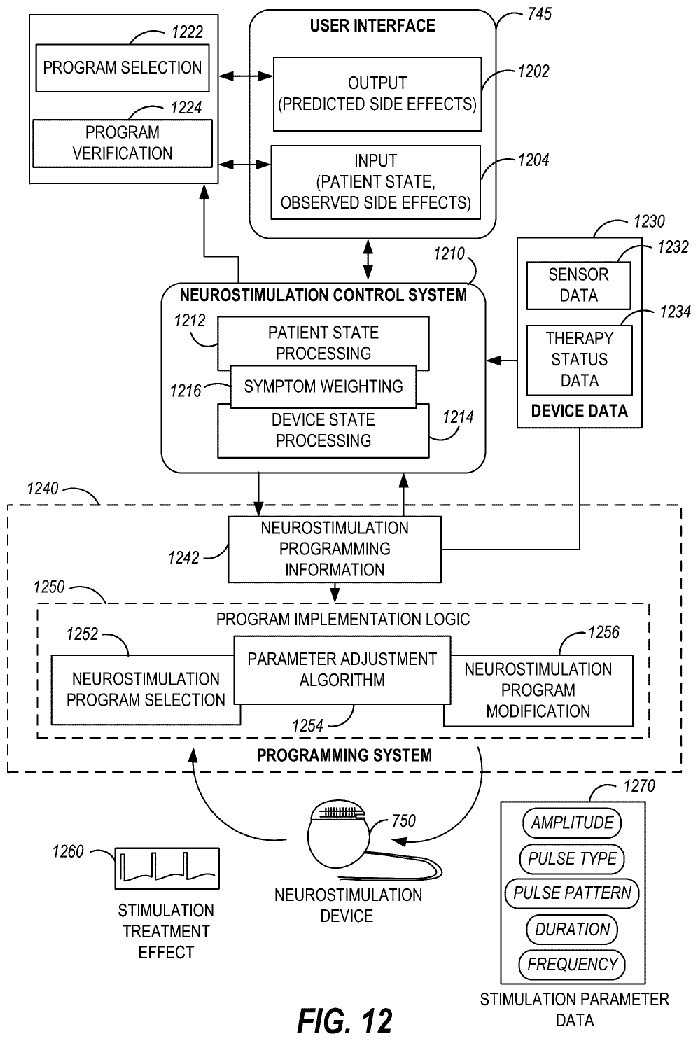

Finally, the patient data analysis system 650 optionally includes a neurostimulation programming engine 714 to evaluate or determine operational conditions of programming for the neurostimulation device 750, based on the modeled side effects, patient symptoms and treatment objectives, and user inputs/commands related to the testing of neurostimulation programming. In an example, the neurostimulation programming engine 714 provides control, modification, selection, or specification of neurostimulation programming parameters, in an automatic, suggested, or manual fashion, including based on a calculation of a lack of predicted side effects, or confirmation by a patient, clinician, or caregiver that observed side effects can be safely handled by the patient. Additional detail regarding closed-loop programming or control of the device 750 based on predicted or observed side effects is provided in the example of FIG. 12, below, but it will be understood that other embodiments of side effect evaluation and prediction, and program modeling, selection, recommendation, and implementation may be provided via programming devices, data services, or information services that are not depicted.

In some examples, the clinician computing device 730 or patient computing device 740 may provide data used for modeling or evaluating the side effects, and may provide outputs based on modeling or evaluating the side effects. Outputs may include questions, surveys, warnings, or information about predicted side effects. Inputs may include patient state information, clinical effects data observations and data values, programming parameter and parameter value selections, programming exploration commands, and the like.

In an example, the patient computing device 740 is a computing device (e.g., personal computer, tablet, smartphone) or other user-operated device that receives and provides interaction with a patient or their caregiver using a graphical user interface 745. Within the graphical user interface 745, input functionality is provided through a programming setting selection functionality 742 and patient status functionality 744. For instance, the programming setting selection functionality 742 may receive a selection or modification of neurostimulation programming parameters by a patient (e.g., a new program selection, the acceptance of a recommended setting, a manually entered value associated with a programming parameter, etc.). The patient status functionality 744 may be used to receive input (e.g., via text, answer fields, prompts, or other user input) that indicate or confirm whether one or more predicted side effects have been observed, characteristics of the observed side effects, and information related to medical condition symptoms or treatment outcomes.

The clinician computing device 730 likewise may be a computing device which implements a graphical user interface 735. The features of the graphical user interface 735 may offer similar capabilities to the graphical user interface 745 provided to the patient, but adapted for use by a clinician or other medical professional (e.g., to provide enhanced functionality or features for control and input by a physician, nurse, medical device representative, etc.). Such functionality may include programming setting selection functionality 732, patient status functionality 734, and clinical data functionality 736. For instance, the programming setting selection functionality 732 may be used to select or schedule programming parameters to be tested or implemented; patient status functionality 734 may be used to enter or track patient status values relevant to neurostimulation treatment side effects or patient symptoms; and clinical data functionality 736 may be used to input, select, or identify clinical data fields relevant to neurostimulation treatment side effects or underlying medical condition symptoms, consistent with the examples herein.

Some data values and neurostimulation programming may be automatically determined or adjusted based on closed-loop or patient-responsive (i.e., partially-closed-loop) programming changes, including based on symptom or side effect states (including detected and predicted states). For instance, the patient data analysis system 650 may utilize sensor data 760 from one or more patient sensors 770 (e.g., wearables, sleep trackers, motion tracker, implantable devices, etc.) among one or more internal or external devices. The sensor data 760 may provide medical data to determine a customized and current state of the patient condition or neurostimulation treatment results, including states that are correlated or associated with side effects. In various examples, the neurostimulation device 750 includes sensors which contribute to the sensor data 760 evaluated by the patient data analysis system 650.

In an example, the patient sensors 770 are physiological or biopsychosocial sensors that collect data relevant to physical, biopsychosocial (e.g., stress and/or mood biomarkers), or physiological factors relevant to a state of the patient. Examples of such sensors might include a sleep sensor to sense the patient's sleep state (e.g., for detecting lack of sleep), a respiration sensor to measure patient breathing rate or capacity, a movement sensor to identify an amount or type of movement, a heart rate sensor to sense the patient's heart rate, a blood pressure sensor to sense the patient's blood pressure, an electrodermal activity (EDA) sensor to sense the patient's EDA (e.g., galvanic skin response), a facial recognition sensor to sense the patient's facial expression, a voice sensor (e.g., microphone) to sense the patient's voice, and/or an electrochemical sensor to sense stress biomarkers from the patient's body fluids (e.g., enzymes and/or ions, such as lactate or cortisol from saliva or sweat). Other types or form factors of sensor devices may also be utilized.

FIG. 8 illustrates, by way of example, an estimation of neurostimulation side effects based on data value modeling from clinical effects. The data plot of FIG. 8 shows how data values are mapped relative to a first dimension of data (lead level values, charted on first axis 810) and a second dimension of data (amplitude values, charted on second axis 820). Here, only two dimensions of data are depicted, but it will be understood that three, four, or many more dimensions of data may be mapped and considered. Various data manipulations (such as data rotation in a three-dimensional data mapping space) may also apply to one or more dimensions of the data.

Within the example of FIG. 8, a set of data points 830 (lightly patterned in the data plot) are mapped to represent data values where adverse side effects have not occurred, and a set of data points 840 (darkly patterned in the data plot) are mapped to represent data values where adverse side effects have occurred. Here, a new data point 801 is also mapped to represent new programming settings with corresponding lead level values and amplitude values that have not been tested. An evaluation of the new data point 801 relative to the data points 830, 840 may be performed after a sufficient amount of clinical effects data has been captured. Such clinical effects may be directly captured from the patient or from a patient population from the use of specific programming parameters and parameter values—with at least some of the data points capturing detailed side effects.

In an example, the evaluation of the data points may identify a most likely side effect (or effects) that may occur based on data value proximity or closeness. This is shown with the evaluation of a new programming setting (involving lead level and amplitude values) corresponding to the new data point 801. Here, the closest side effects in this data plot have been observed at data points 802A and 802B. This data modeling suggests that that the most probable side effects that may occur are those corresponding to the side effects experienced using values at data point 802A (the closest data point) or values at the data point 802B (the second-closest data point).

In further examples, data values for multiple side effects that are nearby an evaluated data point (e.g., nearby the new data point 801) can be considered and ranked. For example, a decision algorithm can be used to incorporate different probabilities of side effects to create a ranking/probability system for determining a likelihood of possible side effects. Additional dimensions and types of data may be considered, as the consideration of nearby data values in a mapped data modeling space is not limited to two dimensions or to consideration of only lead level values or amplitude values.

FIG. 9A illustrates an estimation of neurostimulation side effects based on stimulation field modeling (SFM) by using neural activation models and anatomical modeling data. Here, the neurostimulation of specific anatomical area, shown in treatment modeling area 922, can be modeled to produce a known treatment effect based on the location of one or more electrodes on a lead 911.

Neurostimulation of a nearby anatomical area can also be modeled to predict a known side effect, shown in side effect modeling area 923. For example, consider the side effects from DBS of the brain area surrounding the subthalamic nucleus (STN) for treatment of Parkinson disease, documented by Szczakowska et al., Deep Brain Stimulation in the Treatment of Tardive Dyskinesia, J. Clin Med. (2023 March; 12(5): 1868):

TABLE 1

| Side Effect | Brain Area |
|---|---|
| Spastic muscle | Internal capsule |
| contraction | |
| Uni- or bilateral | Fibers stemming from the frontal eye field running in the internal capsule, |
| gaze deviation | fibers of the third nerve (inferomedial to the STN and within the red |
| | nucleus), sympathetic fibers within the zona incerta or STN |
| Autonomic | Hypothalamus and red nucleus |
| symptoms | |
| Paresthesia | Medial lemniscus |
| Speech | Internal capsule, the pallidal and cerebello-thalamic fiber tracts medial and |
| impairment | dorsal of the STN, medial left-sided STN stimulation in right-handed |
| | patients, higher left STN voltage |
| Depression | Substantia nigra |
| Mania | Medial and ventral areas of STN |
| Impulse control | Ventromedial and limbic areas of STN, SNr, medial forebrain bundle |
| disorder | |
| Cognitive | Ventral and medial parts of STN, perforation of the caudate nucleus during |
| problems | surgery |

One or more side effects can be predicted and monitored when neurostimulation effects as predicted by a stimulation field modeling volume (such as the treatment modeling area 922) overlaps or is close to specific anatomical structures or regions associated with a given side effect modeling volume (such as the side effect modeling area 923). As the stimulation volume approaches a proximity of the side effect regions, data modeling can be used to identify that the nearest side effect regions may be possibly activated-even if overlap is not shown. Once a threshold of closeness is achieved and the side effect is identified as a “predicted”, “probable”, or “possible” side effect, then the presence or absence of these side effects may be monitored and recorded during testing.

Regions that are the most overlapped or the closest to the stimulation field modeling results can be prioritized if multiple regions of treatment effects or side effects are identified. This may lead to the identification of a most likely side effect or multiple likely side effects. Prior mapping data or general knowledge of functional neuroanatomy can further guide this process. For instance, if clinical effects data and anatomical mapping data are available, both sets of data can be combined or evaluated in a weighted manner. As one example, predicted side effects from clinical effects data can be given more weighting than predicted side effects from general anatomical modeling.

In further examples, more general anatomical data may be imported and considered. For instance, certain structures/regions may be pre-labeled as side effect structures, and types of side effects anticipated from these structure/regions can be pre-assigned based on literature or aggregated analysis. Also in some examples, higher amplitude settings or other high-intensity values of neurostimulation may also regarded as overstimulation side effects, and can be treated as a side effect structure.

FIG. 9B illustrates, by way of example, an estimation of neurostimulation side effects by activation of fiber tracts (a pathway of one or more neural fibers) based on stimulation field modeling. If fiber tracking data is available in addition to basic anatomical modeling data, then the side effects associated with activation of those fiber tracts can also be considered. This enables the stimulation as predicted by a stimulation field modeling volume, such as stimulation that activates fiber tracts that reach structures or regions distant to the implanted lead and associated with a given side effect.

The activation of neural fibers may be assessed in different ways. In one example, the identification of a neural fiber that intersects with a given stimulation field modeling area can be considered as activated or triggered by stimulation. This is shown in FIG. 9B, where a treatment modeling area 924 is modeled to primarily produce a known treatment effect based on the location of one or more electrodes on a lead 921. Additionally, the position of the lead 921 suggests a possible effects area 925 that overlaps the location of one the fibers 930 connected to a side effect modeling area 926. Thus, the position of the lead 921 can be identified as possibly activating a neural fiber that triggers the side effect associated with the side effect modeling area 926. In further examples, stimulation field modeling can also evaluate specific characteristics of a given fiber, such as fiber diameter, curvature, etc.

The information determined from the preceding side effect predictions can be used in a variety of scenarios. For example, workflows may also enhance testing of new programming values in a clinical setting. As one example, after side effects are predicted, a physician may be queried during in person or remote programming via a clinical programmer to look for specific side effects based in a given stimulation region. Predicted side effects can also be used to identify the characteristics of the side effect, such as to warn the physician if a particular side effect is possible to occur but will not be observable (e.g., wash-in time) until more than 5 minutes after starting stimulation.

As another example, side effect prediction may be used as a mechanism to fully test out a new stimulation setting (or, to continue testing of a new stimulation setting) in an in-home setting, even if the new stimulation setting has been used for less than the predicted wash-in time for at least one of the anticipated side effects. The patient can be queried after stimulation is implemented at one or multiple time points (e.g., at expected short-term or long-term wash-in time points associated with the side effects), to verify whether adverse or problematic side effects have occurred.

In still further examples, the present side effect functionality may allow a stimulation setting to be tested or evaluated under different medication conditions, because the use of medication may affect the amount, timing, or type of side effects. Thus, medication state may be considered in addition to other real-time patient state information to determine the possible level or characteristics of one or more side effects.

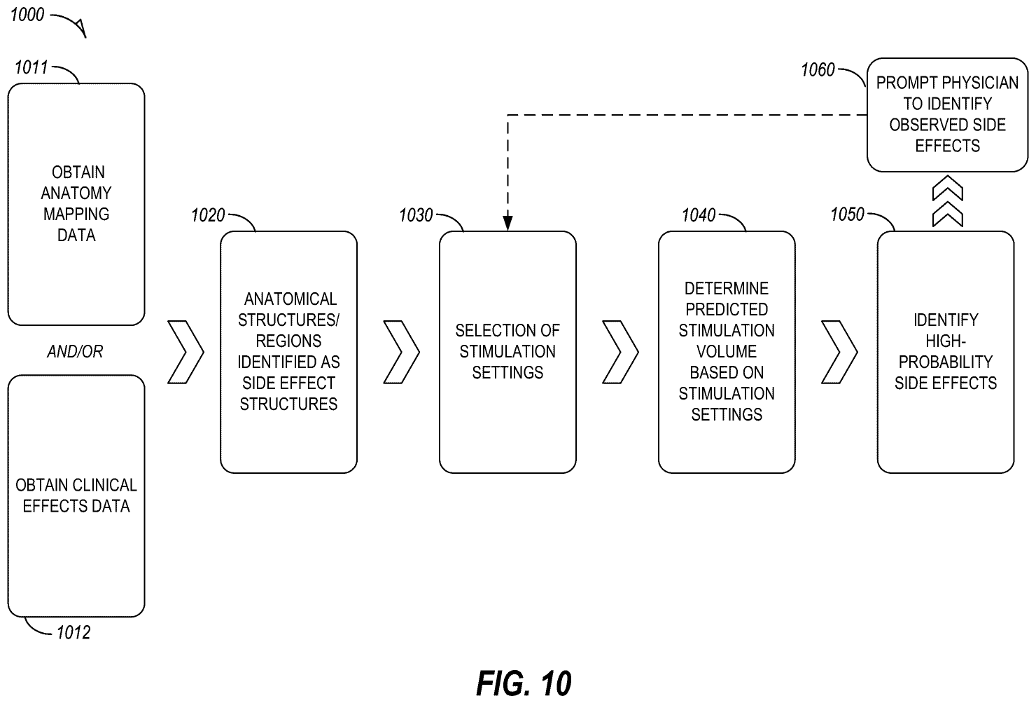

FIG. 10 illustrates, by way of example, a data processing flow 1000 for side effect monitoring of neurostimulation treatment in a clinical setting, such as by use of a physician or other medical professional to begin a testing of neurostimulation programming parameters. Here, the flow 1000 begins by obtaining source data that defines the effects of the neurostimulation treatment in some anatomical area of a patient. This may include obtaining anatomy mapping data at operation 1011 (e.g., atlases that map side effects or treatment effects to particular anatomical areas), obtaining clinical effects data at operation 1012 (e.g., data measurements that map side effects or treatment effects to particular data values), or both.

The anatomical structures/regions are modeled at operation 1020, to identify side effect structures or regions that are relevant to various anatomical areas. For instance, anatomy mapping data that is known to provide a particular side effect in a specific anatomical area may be correlated to specific characteristics of the particular side effect as indicated by clinical effects data. For instance, the clinical effects data may show that side effects are likely to emerge for the patient in the specific anatomical area at a particular time.

A selection of stimulation settings is made by the clinician or another medical professional at operation 1030. This selection of stimulation settings may include neurostimulation programming values that are determined from parameter exploration or modification (including closed-loop programming recommendations), and which have not been tested on any patient or tested on the particular patient. This selection of stimulation settings may be provided as further discussed in FIG. 12, below.

A predicted stimulation volume is determined from the stimulation settings at operation 1040. This predicted stimulation volume may be produced from stimulation field modeling, using any number of techniques or approaches. The predicted stimulation volume may be compared with known side effects data to predict high-probability side effects at operation 1050. This may include evaluation of data values associated with the predicted stimulation volume and side effects, or overlapping/proximate spaces between the predicted stimulation volume (i.e., where stimulation effects occur) and a predicted side effects volume (i.e., where stimulation side effects are triggered). This may be performed based on the modeling approaches described for FIGS. 9A and 9B.

A clinician (e.g., physician, nurse, medical professional, device manufacturer representative) be prompted at operation 1060 to confirm, deny, or record results of the predicted side effects, or to provide related notifications and receive information for at least side effect. In some examples, this may include a warning if the side effect is possible to occur but has a wash-in time greater than some value (e.g., 5 minutes). Based on information collected in this prompt, the neurostimulation programming values may be enabled or scheduled in a program (e.g., if adverse side effects are not observed by the clinician), or may be disabled or prevented from use (e.g., if adverse side effects are observed by the clinician).

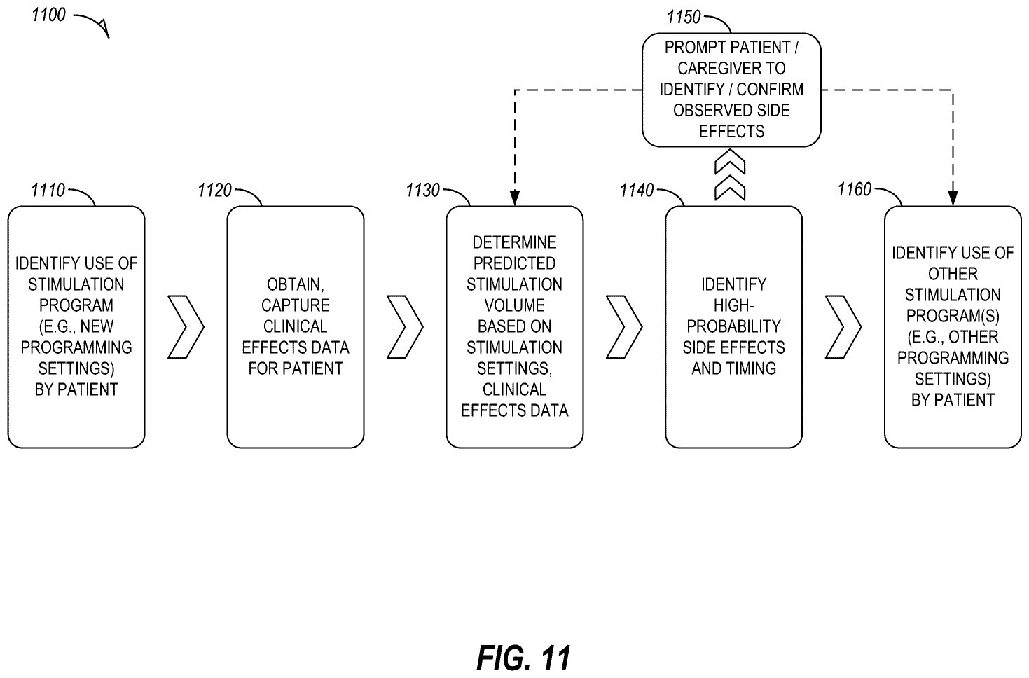

FIG. 11 illustrates, by way of example, a data processing flow 1100 for side effect monitoring of neurostimulation treatment in a patient setting. The flow 1100 begins with a use of stimulation program (e.g., new programming settings) by a patient at operation 1110, in a testing or operational use scenario. Such a scenario may occur at home, or may occur under the supervision of a caregiver or medical professional.

Clinical effects data for the patient is obtained and/or captured at operation 1120, including historical side effects data captured from the use of various sensor devices or patient feedback. The clinical effects data may include patient data from partially testing a particular program or set of programming values in a monitored setting.

A predicted stimulation volume is determined at operation 1130 based on use of the stimulation program (e.g., partial testing results from in-clinic testing), clinical effects data that includes related historical side effects, and other anatomical data. This predicted stimulation volume may be produced from stimulation field modeling, as discussed above, including the modeling approaches described for FIGS. 9A and 9B.

The modeling is used to produce data that identifies a high-probability side effects and timing at operation 1140. Each side effect may be identified with an estimated wash-in time. In some examples, a pre-set time may be used, which can be adjusted manually or automatically based on recorded observations from the patient. If a pre-set time is used and there are side effects that wash in later, the user can be prompted at a later time (e.g., a second time) to identify if they are experiencing any of the potential side effects. Additional warnings or programming changes can also be used if the program exceeds a threshold of closeness to a particular type or intensity of a side effect.

Based on the probability, timing, and other characteristics of the side effects, the patient (or a caregiver, or medical professional) may be prompted to identify or confirm observed side effects at operation 1150. Also, based on the confirmation of side effects (or lack of side effects), additional testing or remedial actions may occur. Remedial actions may include the use of other (e.g., alternative) stimulation program(s) and other programming settings at operation 1160, clinical intervention, or a return to known acceptable settings.

FIG. 12 illustrates, by way of example, an embodiment of a closed-loop or partially-closed-loop data processing flow affecting the neurostimulation treatment of a patient, which may be coordinated with the side effect prediction and monitoring operations discussed above. Specifically, this data processing flow shows how a neurostimulation control system 1210 may include patient state processing functions 1212 and device state processing functions 1214, based on predicted and observed side effects (including predicted and observed effects as discussed above) to control neurostimulation programming. Other user interfaces and functionality are not depicted for simplicity.

In this example, the user interface 745 (e.g., a patient user interface), is used to obtain and provide information to a user (patient, caregiver, clinician or medical professional) related to side effects based on the workflows discussed above. For instance, the user interface 745 may include input functionality 1204 to collect information on the patient state or treatment that is relevant to side effects determination, and output functionality 1202 to provide information relevant predicted side effects or treatment outcomes. The user interface may involve the input functionality 1204 and output functionality 1202 during program selection 1222 (e.g., to enable the use of a program with particular neurostimulation settings, based on side effect prediction), and program verification 1224 (e.g., to verify the activation, scheduling, or use of a program with particular neurostimulation settings, based on side effect prediction). Such operations and functionality may involve the side effect evaluation discussed with reference to FIGS. 7 to 11.

FIG. 12 also depicts the evaluation of device data 1230, such as sensor data 1232, therapy status data 1234, and other treatment aspects which may be obtained or derived from the neurostimulation device 750 or related neurostimulation programming. The device data 1230 and the inputs received with the user interface 745 allow a patient state and device state to be determined within patient state processing functions 1212 and device state processing functions 1214, including to detect or identify states that are relevant to side effect detection or symptom evaluation. Such symptom evaluation may include the following approach for automatically determining symptom prioritization for treatment and programming.

In an example, the user interface 745 obtains the patient specific symptoms of significance via one or multiple of the following: physician identified symptoms (e.g., recorded on the programmer 412 or another connected system); use of patient directed questionnaires collected over time; automated assessments performed on the user interface 745; or assessments of internal or external sensor data provided with device data 1230. Various types of symptom weighting may be performed by the patient state processing functions 1212 and device state processing functions 1214 to determine correct symptoms and weighting for treatment and re-programming. Where data differs between symptom selection methods inclusion priority can be provided in the following order: physician-selected symptoms; patient-identified symptoms; or additional symptoms based on the confidence in the assessment.

Automated testing and evaluation of programming settings may be performed via the neurostimulation control settings, based on side effect prediction and/or symptom weighting approaches. The testing and evaluation of programming settings may be coordinated by symptom weighting functionality 1216, which is used by the neurostimulation control system 1210 to determines which symptoms to prioritize during treatment. As an example, consider the following scenario for in-home usage of patient programming. At a set time prior to a scheduled physician visit, the user interface 745 pushes tasks to a patient to assess the impact of current treatments on patient specific symptoms during treatment low and treatment high periods.

In various scenarios, the identification of symptoms may consider the use of medication, and coordinate treatment strategies based on observed medication states. For example, a medication-only task flow may include assessing selected symptom severity during a medication low period, and reassessing selected symptom severity during a medication high period. A stimulation-only task flow may include assessing selected symptom severity when stimulation is turned on or off (e.g., a long off period). A medication and stimulation task flow may include assessing selected symptom severity during medication low period with stimulation off (e.g., a short off period) and reassessing selected symptoms severity during a medication high period with stimulation on. Medication low and high periods may be determined by medication schedule plus an anticipated wash-in time for effects (e.g., based on literature or expert opinion), or by sensors or medical data observations. Wash in time can be manually or automatically adjusted by the physician or the control system, respectively, if collected data has demonstrated a patient specific wash-in time. Long off in an example may constitute 30 minutes or a patient tolerated off time (whichever comes first). Short off in an example may constitute 5 minutes or less. Repeated testing can be performed (and spaced out) over the course of several days or weeks.

Weighting of particular symptoms may be coordinated by the symptom weighting functionality 1216 in the neurostimulation control system 1210 by comparing the symptom severity during treatment high and treatment low periods. Generally, symptoms that have a larger difference in their treatment high and treatment low evaluations can be given higher weights. Two symptoms tied in weight based on response to medication can be weighted differently based on patient preference toward improving one versus the other (e.g., as determined by a questionnaire response). Symptoms that do not respond to treatment can be given a weight of zero, whereas symptoms that respond negatively to treatment (or, those considered as side effects) can be given a negative weighting. If repeated testing has been performed, results are averaged across the multiple tests that represent the same approximate “timepoint” of data. In various examples, symptoms with high positive weights can be prioritized for use and automatically recommended to the user along with their associated weights. Symptoms reaching a set threshold of negative weighting can be similarly included. Symptoms with high severity (or significance to the patient) but no response to treatment can be prioritized with a flag indicating that the response is not anticipated to respond to treatment (with a weight listed as zero, meaning no impact to the programming algorithm). If the symptom demonstrates response during testing, this flag allows the symptom to adjust the weighting dynamically to reflect the recorded high and low scores.

Other evaluation of activity variability and weighting may be provided based on symptom or side effect weighting. For example, in a scenario where the patient or a sensing device indicates that one or multiple individual activities significantly impact outcomes, weights can be calculated for individual activities. Calculating weights per activity may include: prompting a patient to stay in the same treatment state (no change in medication or stimulation); prompting the patient, without doing the activity to report or automatically record symptoms; prompting a patient to record a moment of maximal symptom impact after the patient begins performing the activity; and the like. The difference in recordings before and during activity can be used to set activity-specific weights and state values.

Various approaches for programming may occur based on use of multiple weight settings derived from symptoms or side effects. If a user has activity variability and is undergoing programming, the non-activity treatment high/low weights can used for initial programming. After completion, the weights from an activity can be used to either select a best setting from the current clinical effects map, or to perform or guide additional exploration as needed.

The remainder of the data processing flow illustrates how the patient state and device state can be used by the neurostimulation control system 1210 to implement programming, such as in a closed loop (or partially-closed-loop) system. A programming system 1240 uses programming information 1242 provided from the neurostimulation control system 1210 as an input to program implementation logic 1250. The program implementation logic 1250 may be implemented by a parameter adjustment algorithm 1254, which affects a neurostimulation program selection 1252 or a neurostimulation program modification 1256. For instance, some parameter changes may be implemented by a simple modification to a program operation; other parameter changes may require a new program to be deployed. The results of the parameter or program changes or selection provides various stimulation parameters 1270 to the neurostimulation device 750, causing a different or new stimulation treatment effect 1260. The new or ongoing use of neurostimulation parameters may be verified for symptoms, side effects, and treatment outcomes.

By way of example, operational parameters of the neurostimulation device which may be generated, identified, or evaluated by the neurostimulation control system 1210 may include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The neurostimulator may use current or voltage sources to provide the neurostimulator output, and apply any number of control techniques to modify the electrical simulation applied to anatomical sites or systems related to pain or analgesic effect. In various embodiments, a neurostimulator program may be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

In still further examples, the approaches of closed-loop programming or human-responsive (i.e., partially-closed-loop) programming may be accompanied by various aspects of health monitoring, medication and patient state tracking, medication or treatment reminders, and other targeted health actions. For instance, medication schedules and reminders can help identify what medication a patient is taking and when a dose was taken, but cannot guarantee that the medication is being taken when the patient is in an ideal state. Some medications should be taken after a meal or should not be taken after consuming a high protein meal. Missed and failed doses of medications present a problem for patients, especially for patients that face cognitive impairments.

The user interface 745, neurostimulation control system 1210 and other components of FIG. 12 can be adapted to assign attributes to medication, so that specific dietary or medication reminders are scheduled to be sent to the patient based on the estimated impact on a medication's efficacy. Targeted health monitoring can also be used to identify or mitigate potential dietary interactions with current medications, and to coordinate with neurostimulation.

Consider the following complications when attempting to coordinate medication intake for treatment of Parkinson disease that is provided in addition to neurostimulation. Certain medications are less effective when taken with certain foods. Carbidopa and levodopa should not generally be taken with high protein intake and are suggested to be taken on an empty stomach (e.g., 30 minutes before and 1-2 hours after a meal), otherwise, the medication may not be absorbed as effectively. Other medications can lead to additional side effects when taken under different dietary conditions. Medications such as bromocriptine, ropinirole, benztropine mesylate, and amantadine should not be taken with alcohol as it can increase cognitive impairment. Monoamine oxidase inhibitors (MAIOs) should not be taken with foods high in tyramine because this can cause a spike in blood pressure gastric state, which modified by food consumption, can affect medication absorption and timing and may be related to failed dosing. In an example, specific dietary conditions that are associated with a medication can be pre-assigned and presented in the user interface 745. These conditions and related recommendations/guidance also can be assigned by the patient, or can be assigned by a physician or caregiver that has access to the system.

The evaluation of side effects and patient state in the neurostimulation control system 1210 may consider other aspects of patient activity and behavior, including dietary information and related activities. Dietary information may be recorded, tracked, and also output in the user interface 745 in connection with recommendations or schedules. In an example, recording dietary intake may include asking a patient to indicate when they eat via any of the following mechanisms: input on a software app; a button click on an external device; an input on the IPG or on another medical device. Dietary intake can also be estimated by recorded data obtained from a sensing device, including using signals from implanted and non-implanted devices. Non-implanted devices could include, for example, glucose monitoring from a continuous glucose sensor. In other examples, the patient may set a schedule of when they plan to eat. A wearable device or phone app can provide a notification or prompt a user to verify the event (e.g., 1 hour or another period of time after the scheduled time). A pattern of eating behavior also can be estimated based on any single or multiple data points of the above and verified by a confirmatory signal that can be user input or reliable sensed data. In other examples, estimated eating behavior can be used in conjunction with scheduled or human-recorded inputs.

This information and other sensed signals can be used to identify a potential dietary impact to treatment provided by medication and neurostimulation. Sensed signals can provide an indication of maximal effectiveness of treatment, which can also indicate the wash-in time for a given medication. Sensed signals may also be used to identify missed and/or failed dosing. For each patient, an average wash-in time estimate can be determined based on time from known or scheduled medication dose to maximal effectiveness. Based on the estimated medication times and activities (discussed above), and if there is a significant delay in the time until maximal effectiveness, a flag or alert can be set to indicate a potential interference from diet.

Other aspects of the user interface 745 may include various medication or dietary timers, reminders, schedules, and monitoring. For instance, patients can receive medication reminders on their mobile devices to help track when they need to take their various scheduled medications. For medications that require a patient to be in a given dietary state, one of the following approaches can be used. Based on the patient indicating that they have eaten, a scheduled medication can be automatically adjusted. In the case of an upcoming treatment which is restricted by eating (e.g., carbidopa/levodopa), the medication schedule can be automatically delayed for 1 hour after eating. In the case of an upcoming treatment with other medications (e.g., MAOIs), the user may be asked if they have eaten any of a certain type of food, and if so then their dose may be delayed. An additional reminder can be set to help ensure they are in the correct dietary state for their medication. For example, 30 minutes before a patient is scheduled to take carbidopa/levodopa, they may receive a notification via the user interface 745 such as, “Remember don't eat for the next 30 minutes, your next dose of Carbidopa/levodopa is coming up!” Then, when a timer is triggered for their medication, the patient can be reminded or asked if they have eaten in the last 30 minutes (and, if they have, the system can recommend delaying the Carbidopa/levodopa dose by 1 hour and upon acceptance set a new medication timer). Other sensed signals coordinated with the device data 1230 (e.g., sensor data 1232, therapy status data 1234) may be used to adjust recommended timing, notify the patient of a need to adjust stimulation or medication, or flag a user for a physician/clinician consultation.

For general dietary restrictions, a regular reminder (e.g., once a month) can be sent to the patient or caregiver to remind them to avoid or limit consumption of certain foods. If it is impractical to ask patients to regularly enter their diet when taking a medication with a known dietary interaction, then additional monitoring for interactions can be performed. Examples include: For Carbidopa/levodopa, if reduced efficacy is regularly seen in the dose taken around a scheduled meal (or an estimated or observed meal), a schedule change may be suggested to move the administration to longer before or after eating. For patients taking MAOIs, the patient may be encouraged to utilize a device capable or automatically capturing blood pressure (e.g., via other sensors). If regular high blood pressure readings are identified on dosing occurring near meal times, a schedule change may be suggested to move the administration to longer before or after eating. In this case, the patient, physician, and caregiver can be informed of the potential interaction.

Patient state information used by the patient state processing functions 1212 also can be identified using multiple data streams of these and other information, including user inputs to devices (the user interface 745, apps on user phones, apps on other devices, multi-sensor watches, other user-input or user-tracking sensors). This may handle situations such as: a missed dose where a patient does not ingest their medications; a non-response (a failed dose) where the patient ingests the medications but the intended effect doesn't occur; an affected dose, where gastric state, foods, or drugs consumed interact with the medication of interest to alter the therapy state (e.g. by accelerating/delaying effects of medication, or boosting/inhibiting effects of medication). These states may be detected and used to trigger notifications to the patient, the patient's clinical care team, clinicians, or device managers. Adjustments can be made to enhance therapy as part of the neurostimulation or the medication strategy, including but not limited to dosing amount, frequency, or type.

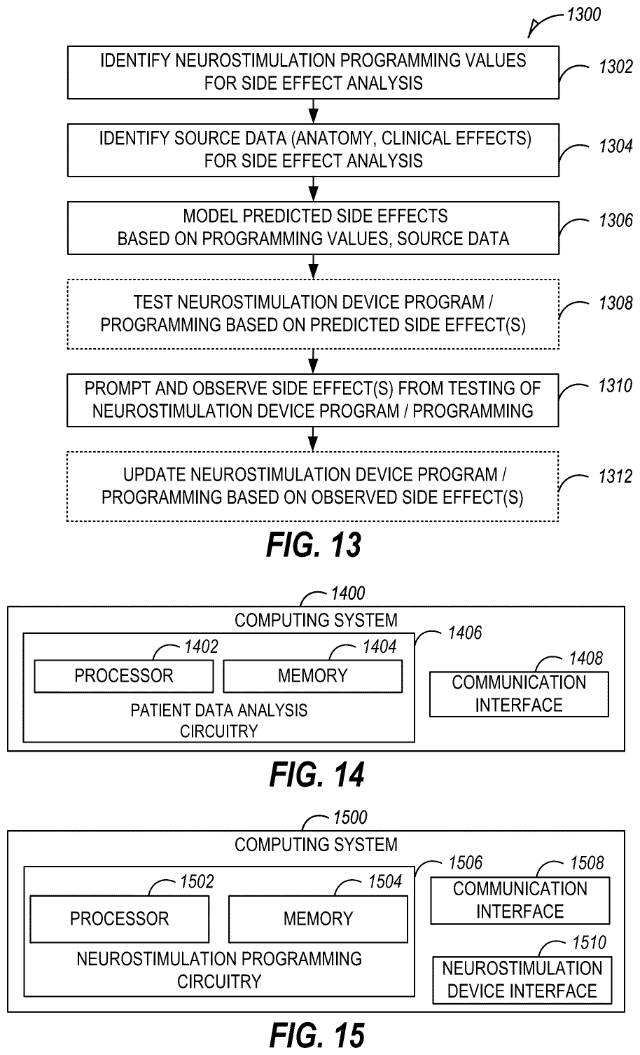

FIG. 13 illustrates, by way of example, an embodiment of a processing method 1300 implemented by a system or device for use to identify side effects or outcomes relating to a neurostimulation treatment. For example, the processing method 1300 can be embodied by electronic operations performed by one or more computing systems or devices (including those at a network-accessible remote service) that are specially programmed to implement the data analysis and/or neurostimulation data processing operations described herein. In specific examples, the operations of the method 1300 may be implemented through the systems and data flows depicted above in FIGS. 6 to 12, at a single entity or at multiple locations.

In an example, the method 1300 begins at operation 1302 by identifying (e.g., obtaining, retrieving, calculating) neurostimulation programming parameters (e.g., parameter values) for side effect analysis. These neurostimulation programming parameters are to be used in a neurostimulation device of a patient (or, neurostimulation devices of a patient group or population of patients). Consistent with the examples above, the neurostimulation programming parameters or values may relate to timing, amplitude, frequency, intensity, duration, pulse patterns, pulse shapes, a spatial location of pulses, waveform shapes, or a spatial location of waveform shapes, of modulated energy provided with at least one lead of the neurostimulation device.

The method 1300 continues at operation 1304 by identifying (e.g., obtaining, retrieving, deriving) source patient data (e.g., clinical effects data, anatomy data) for side effect analysis. This source data may define effects of neurostimulation treatment in a specific anatomical area (or areas) of the patient. In one example, the source data includes clinical effects data collected from the patient, and the clinical effects data includes observed data values mapped in multiple dimensions corresponding to neurostimulation parameters. This observed data enables a predicted side effect to be identified based on a proximity or similarity of data values of the neurostimulation programming parameters to the observed data values mapped in the multiple dimensions.

The method 1300 continues at operation 1306 by modeling one or more predicted side effects based on the programming values and the source patient data. The modeling may also be used to produce information that identifies the characteristics of the predicted side effect, such as a type and severity of the predicted side effect and a timing of the predicted side effect. In an example, the operations to model use of the neurostimulation programming parameters include determining a predicted stimulation volume based on modeled treatment effects of the neurostimulation programming parameters in the anatomical area (e.g., as indicated by stimulation field modeling), determining a predicted side effect volume based on modeled side effects of the neurostimulation programming parameters in the anatomical area (e.g., as indicated by the source data), and identifying a predicted side effect or effects based on proximity or overlap of the predicted stimulation volume and the predicted side effect volume. In a specific example, a predicted side effect may be selected as a most likely side effect from among a plurality of possible side effects based on the proximity or overlap of the predicted stimulation volume and the predicted side effect volume. In other examples, more than one predicted side effect may be identified and modeled.

In a specific example the source data includes anatomical mapping data that maps the predicted side effect volume to the information that identifies the predicted side effect(s). Also in a specific example, the anatomical area considered for modeling includes a pathway of neural fibers, and the predicted side effect volume corresponds to a neural fiber, enabling the side effect(s) to be predicted based on a proximity of a stimulation lead to the neural fiber (e.g., for a stimulation lead used to provide the neurostimulation treatment). Any of the modeling and mapping techniques discussed with reference to FIGS. 8 to 11 may be integrated with these operations.

The method 1300 continues at operation 1308 by optionally testing the neurostimulation device program or device programming, to identify whether the predicted side effect(s) have occurred (i.e., are observed side effects). In a further example, this may be implemented by programming of the neurostimulation device with the neurostimulation programming parameters, with the neurostimulation programming parameters being determined based on weights that prioritize treatment of at least one symptom associated with the anatomical area. For instance, the programming may be associated with a test or trial period of the neurostimulation programming parameters in a program of the neurostimulation device.

The method 1300 continues at operation 1310 by observing the side effects from testing neurostimulation device program or device programming. This may be accompanied by (followed or preceded by) a prompt to a medical user associated with the patient, a prompt to a caregiver associated with the patient, or a prompt to the patient. The prompt may include the information that specifically identifies, describes, or suggests the predicted side effect. The prompt may also be accompanied by functionality to collect information relating to an observed side effect (e.g., based on usage of the neurostimulation programming parameters).

In further examples, the method 1300 continues at operation 1312 by optionally updating the neurostimulation device program or device programming, based on the observed side effects. This may include performing a comparison of the predicted side effect to the observed side effect. Consequently, the programming of the neurostimulation device may be updated, based on results of the comparison of the predicted side effect to the observed side effect. This update to the programming of the neurostimulation device may include use of other programming parameters or programs of the neurostimulation device, consistent with the examples above.

FIG. 14 illustrates, by way of example, a block diagram of an embodiment of a system 1400 (e.g., a computing system) for performing patient data analysis in connection with the side effects identification and observation operations discussed above. The system 1400 may be integrated with or coupled to a computing device, a remote control device, patient programmer device, clinician programmer device, program modeling system, or other external device, deployed with neurostimulation treatment. In some examples, the system 1400 may be a networked device (server) connected via a network (or combination of networks) which communicates to one or more devices (clients) using a communication interface 1408 (e.g., communication hardware which implements software network interfaces and services). The network may include local, short-range, or long-range networks, such as Bluetooth, cellular, IEEE 802.11 (Wi-Fi), or other wired or wireless networks.

The system 1400 includes a processor 1402 and a memory 1404, which can be optionally included as part of patient data analysis circuitry 1406. The processor 1402 may be any single processor or group of processors that act cooperatively. The memory 1404 may be any type of memory, including volatile or non-volatile memory. The memory 1404 may include instructions, which when executed by the processor 1402, cause the processor 1402 to implement side effects prediction and tracking, or to enable other features of the patient data analysis circuitry 1406. Thus, electronic operations in the system 1400 may be performed by the processor 1402 or the circuitry 1406.

For example, the processor 1402 or circuitry 1406 may implement any of the features of the method 1300 (such as operations 1302-1312) to obtain and process neurostimulation programming values, to identify and evaluate anatomy and clinical effects data, to model predicted side effects, and to prompt and observe side effects based on testing of the programming values. It will be understood that the processor 1402 or circuitry 1406 may also implement or control aspects of the logic and processing described above with reference to FIGS. 6-13, for use in a various forms of closed-loop and open-loop device programming or related device actions.

FIG. 15 illustrates, by way of example, a block diagram of an embodiment of a system 1500 (e.g., a computing system) implementing neurostimulation programming circuitry 1506 to cause programming of an implantable electrical neurostimulation device, for accomplishing the therapy objectives in a human subject as discussed herein. The system 1500 may be operated by a clinician, a patient, a caregiver, a medical facility, a research institution, a medical device manufacturer or distributor, and embodied in a number of different computing platforms. The system 1500 may be a remote control device, patient programmer device, program modeling system, or other external device, including a regulated device used to directly implement programming commands and modification with a neurostimulation device. In some examples, the system 1500 may be a networked device connected via a network (or combination of networks) to a computing system operating a user interface computing system using a communication interface 1508. The network may include local, short-range, or long-range networks, such as Bluetooth, cellular, IEEE 802.11 (Wi-Fi), or other wired or wireless networks.

The system 1500 includes a processor 1502 and a memory 1504, which can be optionally included as part of neurostimulation programming circuitry 1506. The processor 1502 may be any single processor or group of processors that act cooperatively. The memory 1504 may be any type of memory, including volatile or non-volatile memory. The memory 1504 may include instructions, which when executed by the processor 1502, cause the processor 1502 to implement the features of the neurostimulation programming circuitry 1506. Thus, the electronic operations in the system 1500 may be performed by the processor 1502 or the circuitry 1506.

The processor 1502 or circuitry 1506 may directly or indirectly implement neurostimulation operations associated with the method 1300, including the use of testing neurostimulation device programming (operation 1308) or updating the neurostimulation device programming (operation 1312). The processor 1502 or circuitry 1506 may further provide data and commands to assist the processing and implementation of the programming using communication interface 1508. It will be understood that the processor 1502 or circuitry 1506 may also implement other aspects of the device data processing or device programming functionality described above with reference to FIGS. 6-13.

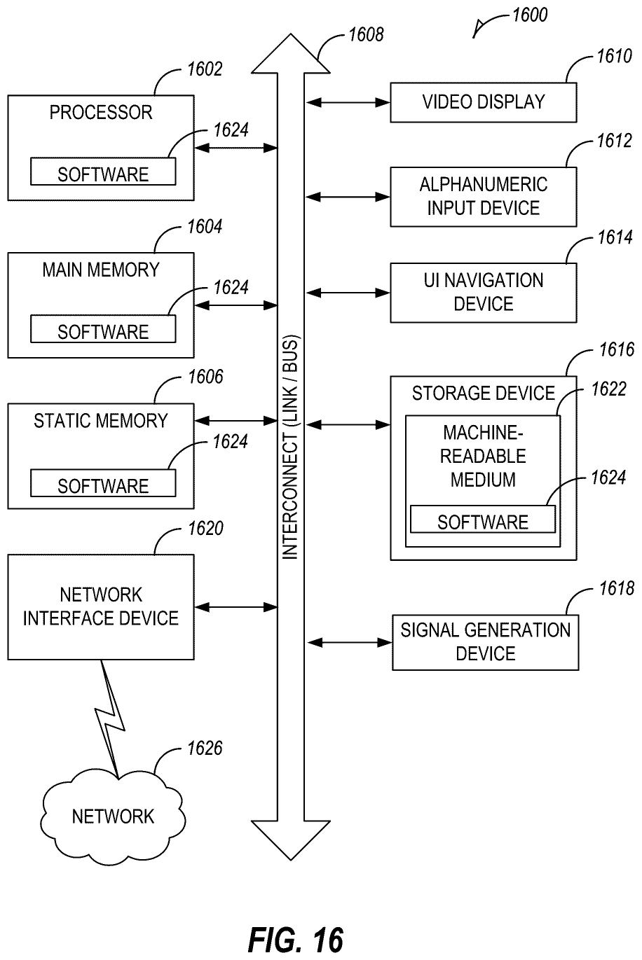

FIG. 16 is a block diagram illustrating a machine in the example form of a computer system 1600, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User's Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1600 includes at least one processor 1602 (e.g., a central processing unit (CPU), a graphics processing unit (GPU) or both, processor cores, compute nodes, etc.), a main memory 1604 and a static memory 1606, which communicate with each other via a link 1608 (e.g., bus). The computer system 1600 may further include a video display unit 1610, an alphanumeric input device 1612 (e.g., a keyboard), and a user interface (UI) navigation device 1614 (e.g., a mouse). In one embodiment, the video display unit 1610, input device 1612 and UI navigation device 1614 are incorporated into a touch screen display. The computer system 1600 may additionally include a storage device 1616 (e.g., a drive unit), a signal generation device 1618 (e.g., a speaker), a network interface device 1620, and one or more sensors (not shown), such as a global positioning system (GPS) sensor, compass, accelerometer, or other sensor. It will be understood that other forms of machines or apparatuses (such as PIG, RC, CP devices, and the like) that are capable of implementing the methodologies discussed in this disclosure may not incorporate or utilize every component depicted in FIG. 16 (such as a GPU, video display unit, keyboard, etc.).

The storage device 1616 includes a machine-readable medium 1622 on which is stored one or more sets of data structures and instructions 1624 (e.g., software) embodying or utilized by any one or more of the methodologies or functions described herein. The instructions 1624 may also reside, completely or at least partially, within the main memory 1604, static memory 1606, and/or within the processor 1602 during execution thereof by the computer system 1600, with the main memory 1604, static memory 1606, and the processor 1602 also constituting machine-readable media.

While the machine-readable medium 1622 is illustrated in an example embodiment to be a single medium, the term “machine-readable medium” may include a single medium or multiple media (e.g., a centralized or distributed database, and/or associated caches and servers) that store the one or more instructions 1624. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1624 may further be transmitted or received over a communications network 1626 using a transmission medium via the network interface device 1620 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

The above detailed description is intended to be illustrative, and not restrictive. The scope of the disclosure should, therefore, be determined with references to the appended claims, along with the full scope of equivalents to which such claims are entitled.

What is claimed is:

1. A device to identify a side effect relating to neurostimulation programming, the device comprising:
one or more processors; and
one or more memory devices comprising instructions, which when executed by the one or more processors, cause the one or more processors to:
obtain neurostimulation programming parameters to be used in a neurostimulation device of a patient;
obtain source data that defines effects of neurostimulation treatment in an anatomical area of the patient;
model use of the neurostimulation programming parameters in the anatomical area of the neurostimulation treatment, based on the source data, to determine a predicted side effect; and
output information that identifies characteristics of the predicted side effect.

2. The device of claim 1, wherein the characteristics of the predicted side effect includes a type and severity of the predicted side effect and a timing of the predicted side effect.

3. The device of claim 1, wherein the source data includes clinical effects data collected from the patient, wherein the clinical effects data includes observed data values mapped in multiple dimensions corresponding to neurostimulation parameters, and wherein the predicted side effect is identified based on a proximity or similarity of data values of the neurostimulation programming parameters to the observed data values mapped in the multiple dimensions.

4. The device of claim 1, wherein to model use of the neurostimulation programming parameters includes to:
determine a predicted stimulation volume, based on modeled treatment effects of the neurostimulation programming parameters in the anatomical area indicated by stimulation field modeling;
determine a predicted side effect volume, based on modeled side effects of the neurostimulation programming parameters in the anatomical area indicated by the source data; and
identify the predicted side effect based on proximity or overlap of the predicted stimulation volume and the predicted side effect volume.

5. The device of claim 4, wherein the predicted side effect is selected as a most likely side effect from among a plurality of possible side effects based on the proximity or overlap of the predicted stimulation volume and the predicted side effect volume; and
wherein the source data includes anatomical mapping data that maps the predicted side effect volume to the information that identifies the predicted side effect.

6. The device of claim 5, wherein the anatomical area includes a pathway of neural fibers, wherein the predicted side effect volume corresponds to a neural fiber, and wherein the side effect is predicted based on a proximity of a stimulation lead to the neural fiber, the stimulation lead used to provide the neurostimulation treatment.

7. The device of claim 6, wherein the instructions further cause the one or more processors to:
cause programming of the neurostimulation device with the neurostimulation programming parameters;
wherein the neurostimulation programming parameters are determined based on weights that prioritize treatment of at least one symptom associated with the anatomical area; and
wherein the programming is associated with a test of the neurostimulation programming parameters in a program of the neurostimulation device.

8. The device of claim 1, wherein the instructions further cause the one or more processors to:
provide a prompt to a medical user, a patient, or a caregiver associated with the patient, the prompt including the information that identifies the predicted side effect; and
collect information relating to an observed side effect based on usage of the neurostimulation programming parameters.

9. The device of claim 8, wherein the instructions further cause the one or more processors to:
perform a comparison of the predicted side effect to the observed side effect; and
update programming of the neurostimulation device, based on the comparison of the predicted side effect to the observed side effect.

10. The device of claim 1, wherein the neurostimulation programming relates to timing, amplitude, frequency, intensity, duration, pulse patterns, pulse shapes, a spatial location of pulses, waveform shapes, or a spatial location of waveform shapes, of modulated energy provided with at least one lead of the neurostimulation device.

11. A method for identifying a side effect relating to neurostimulation programming, comprising:
obtaining neurostimulation programming parameters to be used in a neurostimulation device of a patient;
obtaining source data that defines effects of neurostimulation treatment in an anatomical area of the patient;
modeling use of the neurostimulation programming parameters in the anatomical area of the neurostimulation treatment, based on the source data, to determine a predicted side effect; and
outputting information that identifies characteristics of the predicted side effect.

12. The method of claim 11, wherein the characteristics of the predicted side effect includes a type and severity of the predicted side effect and a timing of the predicted side effect.

13. The method of claim 11, wherein the source data includes clinical effects data collected from the patient, wherein the clinical effects data includes observed data values mapped in multiple dimensions corresponding to neurostimulation parameters, and wherein the predicted side effect is identified based on a proximity or similarity of data values of the neurostimulation programming parameters to the observed data values mapped in the multiple dimensions.

14. The method of claim 11, wherein modeling use of the neurostimulation programming parameters includes:
determining a predicted stimulation volume, based on modeled treatment effects of the neurostimulation programming parameters in the anatomical area indicated by stimulation field modeling;
determining a predicted side effect volume, based on modeled side effects of the neurostimulation programming parameters in the anatomical area indicated by the source data; and
identifying the predicted side effect based on proximity or overlap of the predicted stimulation volume and the predicted side effect volume.

15. The method of claim 14, wherein the predicted side effect is selected as a most likely side effect from among a plurality of possible side effects based on the proximity or overlap of the predicted stimulation volume and the predicted side effect volume; and
wherein the source data includes anatomical mapping data that maps the predicted side effect volume to the information that identifies the predicted side effect.

16. The method of claim 15, wherein the anatomical area includes a pathway of neural fibers, wherein the predicted side effect volume corresponds to a neural fiber, and wherein the side effect is predicted based on a proximity of a stimulation lead to the neural fiber, the stimulation lead used to provide the neurostimulation treatment.

17. The method of claim 16, further comprising:
causing programming of the neurostimulation device with the neurostimulation programming parameters;
wherein the neurostimulation programming parameters are determined based on weights that prioritize treatment of at least one symptom associated with the anatomical area; and
wherein the programming is associated with a test of the neurostimulation programming parameters in a program of the neurostimulation device.

18. The method of claim 11, further comprising:
providing a prompt to a medical user, a patient, or a caregiver associated with the patient, the prompt including the information that identifies the predicted side effect; and
collecting information relating to an observed side effect based on usage of the neurostimulation programming parameters.

19. The method of claim 18, further comprising:
performing a comparison of the predicted side effect to the observed side effect; and
updating programming of the neurostimulation device, based on the comparison of the predicted side effect to the observed side effect.

20. The method of claim 11, wherein the neurostimulation programming relates to timing, amplitude, frequency, intensity, duration, pulse patterns, pulse shapes, a spatial location of pulses, waveform shapes, or a spatial location of waveform shapes, of modulated energy provided with at least one lead of the neurostimulation device.
